(19) United States
(12) Patent Application Publication
(10) Pub. No.: US 2026/0297647 A1
(43) Pub. Date: Oct. 1, 2026

(54) CERTIFICATION OF ANIMAL-DERIVED PRODUCT(S) BASED ON DNA-METHYLATION

(71) Applicants: Evonik Operations GmbH, Essen (DE); DEUTSCHES KREBSFORSCHUNGSZENTRUM STIFTUNG DES ÖFFENTLICHEN RECHTS, Heidelberg (DE)

(72) Inventors: Sanjanaa NAGARAJAN, Singapore (SG); Suki ROY, Singapore (SG); Rose WHELAN, Ambrosden Oxfordshire (GB); Florian BÖHL, Neckargemünd (DE); Frank LYKO, Heidelberg (DE); Sina LYKO-TÖNGES, Heidelberg (DE)

(73) Assignees: Evonik Operations GmbH, Essen (DE); DEUTSCHES KREBSFORSCHUNGSZENTRUM STIFTUNG DES ÖFFENTLICHEN RECHTS, Heidelberg (DE)

(21) Appl. No.: 18/992,420

(22) PCT Filed: Jul. 5, 2023
(86) PCT No.: PCT/EP2023/068584

§ 371 Date: Jan. 8, 2025

(30) Foreign Application Priority Data
Jul. 12, 2022 (EP) 22184431.9

(51) Int. Cl. C12Q 1/68

(52) U.S. Cl. CPC C12Q 1/68; C12Q 2600/112; C12Q 2600/154

(57) ABSTRACT

The present invention relates to a method of certifying a test animal-derived product sample, the method comprising the steps of:
(a) determining a test methylation profile of one or more pre-selected methylation sites within the genomic material obtained from the test animal derived product sample; and
(b) comparing the test methylation profile obtained from (a) with a reference methylation profile obtained from a control animal of the same biological taxon of the test animal from which the product sample is derived from, where the control animal is not slaughtered by a single cut across the throat severing both carotid arteries, both jugular veins, both vagus nerves, trachea and/or esophagus and/or the control animal is not bled to death

wherein a difference in the test methylation profile of (a) compared to the reference methylation profile from the control animal, is indicative of the test animal having been slaughtered by a single cut across the throat severing both carotid arteries, both jugular veins, both vagus nerves, trachea and/or oesophagus and/or of the test animal having been bled to death and the test animal-derived product is certified so, and

wherein the pre-selected methylation sites are CpG sites selected from genes or regions of genomic DNA from the control and test animal that shows the highest degree of methylation variation during the training of the method.

### FIELD OF THE INVENTION

The present invention relates to a method of determining certification of an unknown animal derived product sample based on specific panels of genes that provide a source for the generation of DNA methylation profiles which are specific for a distinct certification. In particular, DNA methylation profiling may be used to determine the distinct certification of animal-derived products. The distinct certification may include halal, kosher and the like.

### BACKGROUND OF THE INVENTION

Sustainable food production is presently considered among the globally most important societal needs. As the value chains of the agriculture and aquaculture industries are highly complex, certificates have been established to reinforce consumer relationships and trust. However, certificates are based on audits at specific farms and can be easily tampered by moving livestock from non-certified farms to certified farms. Furthermore, surveillance of sustainable farming practices is imperfect as it is largely limited to audits. This leaves room for fraudulent activities in the industry, so there is an urgent need for a tamper-proof analysis and certification of meat.

Further, there are people in the world that abide by dietary restrictions that relate specifically to a tradition or a religion that they follow. For example, some Jewish people eat only foods that have been deemed Kosher in accordance with Jewish law. Similarly, some people from the Muslim religion may desire to eat only foods that have been deemed Halal in accordance with Islamic law. Other people may have other dietary restrictions that relate to other traditions, religions, ethical codes, or the like. Both Kosher certification (i.e., designation of a food item as Kosher) and Halal certification (i.e., designation of a food item as Halal) of food, including meat involve the conformance with specific laws that relate to the slaughter of animals. For example, Judaic ritual slaughter (Shechita) and Islamic ritual slaughter (Zabihah) require an animal to be cut across the neck with a non-serrated blade in a single clean attempt to sever main blood vessels of the animal, and to drain the blood of the animal. Health, safety and also animal welfare considerations demand that the origins of animal products, and in particular meat products, should be traceable, so that quality assurance audits, and monitoring procedures can be effectively and reliably carried out. There is, however, no present means of accurately and reliably tracing the origins of meat. In particular, outside of trusting the labels found on the meat packets and shops, there is no reliable, effective and easy to use method for distinguishing halal meat from non-halal meat and kosher meat from non-kosher meat. That is to say, although meat may be certified as Kosher (i.e., allowed under the Jewish tradition) or Halal (i.e., allowed under the Islamic tradition), buyers may be unable to determine whether the meat was properly slaughtered based on visual inspection. Moreover, when consuming food at a restaurant, customers may be unable to view an inspection label affixed to packaging of meat prior to preparation of the meat. Furthermore, a customer may be unable to determine a type of animal from which the meat is derived based on visual inspection. This may result in a person who abides by a dietary restriction being hesitant to consume animal products.

Physical conditions and/or chemical conditions of an animal may be altered based on the way the animal has been slaughtered. For example, it is well known that cortisol levels of animals for which Kosher slaughter or Halal slaughter may be performed may be different from cortisol levels of animals that undergo another means of slaughter. Also, different types of meats obtained from different animals may be associated with physical differences and/or chemical differences. For example, proteins from pork meat of a pig may differ from proteins from beef meat of a cow. Differences may also exist in relation to different breeds of a particular type of animal, different cuts of meat of a particular animal, or the like. This may limit an effectiveness of comparing an unknown sample of meat to a set of known reference samples of meat.

Recent studies have shown that DNA methylation patterns of animals contain important information about their rearing conditions. For example, a comparison of genome-wide patterns of methylation and variation at the DNA level revealed that a highly significant proportion of epigenetic variation could be associated with fitness differences and rearing conditions such as captivity in salmon (Le Luyer J et al. 2017 PNAS vol 114, no 49).

Koop et al. and Rhein et al. also separately confirmed that methylation patterns are maintained postmortem and are consistent regardless of the level of decomposition of the sample. One of the only aspects that still affects the methylation pattern of the sample would be the DNA integrity and amount of DNA that is found in the sample (Koop et al., 2021 International Journal of Legal Medicine 135:167-173 and Rhein et al., 2015 Frontiers in Genetics 6:182).

In view of the above, there is an urgent need to provide means for identifying and quality controlling the certification of organisms, in particular food and more particularly animal material derived to satisfy the dietary restrictions relating to a tradition, a religion, an ethical code, or the like followed strictly by people.

### DESCRIPTION OF THE INVENTION

The present invention attempts to solve the problems above by providing a method using DNA methylation patterns to distinguish one type of animal derived product from another type of animal derived product and to be able to determine the means of killing or slaughtering the animal from which the animal derived product derives from and thereby certify the product accurately and reliably. The present invention is based on the finding that the means of slaughtering an animal, results in stress in the internal environment of the animal and this can permanently change the genome of the animal through epigenetics. In particular, the capability to adapt to the environment (i.e. means of slaughtering) and maintain the adapted biological pattern depends on epigenetic mechanisms, including DNA methylation. In particular, the present invention is based on the finding that the method of slaughtering an animal for food consumption may also result in changes in epigenetic mechanisms of the animal, including DNA methylation patterns and these patterns may be passed down to the different products that may derive from the animal.

The inventors have unexpectedly found that this property can be utilized to identify “epigenetic fingerprints” on the genome that are specific to a method of slaughter of not just one animal but possibly all the animals that undergo the same method of slaughter. For example, these ‘epigenetic fingerprints’ may be specific not only for the animal that is slaughtered in a particular way but also for any product that stems from the very animal (an individual). Based on these findings, the present invention provides means to identify the specific method of slaughter of a particular animal, for example rearing animals also known as livestock and poultry from which an animal-derived product comes from. In particular, the method according to any aspect of the present invention may be used to determine if an animal-derived product is derived from an animal that has been slaughtered in a particular way or not. More in particular, the method according to any aspect of the present invention may be used to determine if an animal-derived product is derived from an animal that has been slaughtered by a single cut across the throat severing both carotid arteries, both jugular veins, both vagus nerves, trachea and/or oesophagus and/or if the animal has been bled to death. In this way, the method according to any aspect of the present invention may then be used to accurately and reliably determine the method of slaughter of an animal and then provide and/or confirm the certification of any sample that originates from the animal. Further, the method according to any aspect of the present invention may also be used to identify if any animal-derived product is accurately and reliably certified, particularly where the certification is based on the method of slaughter of the animal.

According to one aspect of the present invention, there is provided a method of certifying a test animal-derived product sample, the method comprising the steps of:
(a) determining a test methylation profile of one or more pre-selected methylation sites within the genomic material obtained from the test animal derived product sample; and
(b) comparing the test methylation profile obtained from (a) with a reference methylation profile obtained from a control animal of the same biological taxon of the test animal from which the product sample is derived from, where the control animal is not slaughtered by a single cut across the throat severing both carotid arteries, both jugular veins, both vagus nerves, trachea and/or oesophagus and/or the control animal is not bled to death

wherein a difference in the test methylation profile of (a) compared to the reference methylation profile from the control animal, is indicative of the test animal having been slaughtered by a single cut across the throat severing both carotid arteries, both jugular veins, both vagus nerves, trachea and/or oesophagus and/or of the test animal having been bled to death and the test animal-derived product is certified so; and wherein the pre-selected methylation sites are CpG sites selected from genes or regions of genomic DNA from the control and test animal that shows the highest degree of methylation variation during the training of the method.

According to another aspect of the present invention, there is provided a method of certifying a test animal-derived product sample, the method comprising the steps of:
(a) determining the methylation status of at least one CpG site within the genomic material obtained from the animal-derived product sample,
(b) comparing the methylation status of the CpG site from (a) with that of a control animal of the same biological taxon of the test animal from which the product sample is derived from, where the control animal is not slaughtered by a single cut across the throat severing both carotid arteries, both jugular veins, both vagus nerves, trachea and/or oesophagus and/or the control animal is not bled to death,

wherein a difference in the test methylation status of (a) in the test animal compared to the CpG site in the control animal is indicative of the test animal having been slaughtered by a single cut across the throat severing both carotid arteries, both jugular veins, both vagus nerves, trachea and/or oesophagus and/or of the test animal having been bled to death and the test animal-derived product is certified so.

According to yet another aspect of the present invention, there is provided a method of certifying a test animal-derived product sample, the method comprising the steps of:
(a) determining the test methylation profile of one or more pre-selected methylation sites within the genomic material obtained from the test animal derived product sample; and
(b) comparing the test methylation profile determined in (a) with a panel of predetermined reference methylation profiles of the same biological taxon of the test animal from which the product sample derives, wherein each of the predetermined reference methylation profiles is specific for a distinctly certified reference animal that
   has been slaughtered by a single cut across the throat severing both carotid arteries, both jugular veins, both vagus nerves, trachea and/or oesophagus; and/or
   bled to death;

wherein if the test methylation profile is significantly similar to one of the predetermined reference methylation profiles, the test animal derived product sample has the same distinct certification as that of the predetermined reference methylation profile and is derived from a test animal that has been slaughtered by a single cut across the throat severing both carotid arteries, both jugular veins, both vagus nerves, trachea and/or oesophagus and/or bled to death and the test animal-derived product is certified so.

As used herein, the term ‘animal-derived product’ refers to products that originate from animals. In particular, the term ‘test animal-derived product’ refers to the sample or subject in question that is to undergo the method according to any aspect of the present invention. These products from animals may include meat and meat products, also including fat, flesh, blood, and lesser-known products, such as isinglass and rennet, poultry products (meat and eggs), dairy products (milk and cheese), and non-food products such as fiber (wool, mohair, cashmere, leather and the like). Animal-derived products may also include products that can be made using animal products (e.g. fat) such as soap, creams and such. In one example, the animal-derived product is meat, eggs, blood, brain, sperm, milk and any other tissue or sample that provides genomic DNA. In particular, the animal-derived product is meat.

The term ‘certification of quality’ refers to a certificate or a confirmation given by designated certification agencies that endorse the quality of a particular animal derived product, including food for use and/or consumption by human beings. The term ‘certification of quality’ is used interchangeably with the term ‘certification’. These certifications are usually found on the packaging of the animal-derived product including food to be consumed and are printed by the manufactures of the products. Examples of certifications of distinct food quality may include ‘Haltungsform’, ‘Tierwohl’, ‘Ohne Gentechnik’, ‘halal’, ‘kosher’, and other safe labels that confirm that a product sold has been prepared in accordance with specific religious or safety regulations. Specifically, the term ‘certification of food quality’ refers to a certificate or a confirmation given by designated certification agencies that endorse the quality, source or means of slaughter of a particular food for consumption by human beings. According to any aspect of the present invention, the certified quality may be a distinct certified food quality, or distinct certification and this may be kosher, non-kosher, halal or non-halal.

In one example, the distinct certified food quality or certification of the sample X according to any aspect of the present invention may be kosher, non-kosher, halal or non-halal. More in particular, kosher or halal refers to the sample X originating from an animal that was slaughtered by a single cut across the throat severing both carotid arteries, both jugular veins, both vagus nerves, the trachea and/or the oesophagus. Even more in particular, the animal is drained of blood.

The term ‘kosher’ used in combination with food according to any aspect of the present invention refers to food that conforms to Jewish dietary regulations of kashrut (dietary law) or food that may be consumed according to halakha (law). Kosher used in relation to meat relates particularly to a manner in which animals are prepared for consumption. According to Jewish tradition, meat may be considered kosher when the meat comes from animals that have been slaughtered according to Jewish law where the animal is killed by a single cut across the throat to a precise depth, severing both carotid arteries, both jugular veins, both vagus nerves, the trachea and the oesophagus, no higher than the epiglottis and no lower than where cilia begin inside the trachea, causing the animal to bleed to death. Such slaughter is to be carried out using a large, razor-sharp knife, which is checked before each slaughter to ensure that it has no irregularities (such as nicks and dents). The slaughter is usually also carried out by a shochet or a rabbi. Kosher meat usually refers to most meats excluding pig. In particular, kosher meat may be selected from beef, chicken, lamb, mutton, goat meat and mixtures thereof. Kosher meat does not include shellfish, which under Jewish tradition is not permitted for consumption. Although Jewish traditions permit consumption of vertebrate fish, since there is no special method of slaughtering vertebrate fish, all vertebrate fish may be considered kosher. Any food or meat that does not fall within the definition of ‘kosher’ will then be considered as ‘non-kosher’.

The term ‘halal’ used in combination with food according to any aspect of the present invention refers to food that conforms to Islamic dietary laws and especially meat processed and prepared in accordance with those requirements. Similar to the way kosher meat is prepared, in Islamic tradition, animals are slaughtered according to Dhabihah where the animal is slaughtered using a cut across the neck with a non-serrated sharp blade in a single clean attempt to make an incision that cuts the front of the throat, oesophagus and jugular veins but not the spinal cord. In addition to the direction, permitted animals should be slaughtered upon utterance of the Islamic prayer Bismillah. The animal must also be drained of blood after the slaughter. The slaughter must be performed by an adult Muslim. Halal meat usually refers to most meats excluding pig. In particular, halal meat may be selected from beef, chicken, lamb, mutton, goat meat and mixtures thereof. Although Islamic traditions permit consumption of shellfish and vertebrate fish, since there is no special method of preparing shellfish, all shellfish and vertebrate fish may be considered halal. Any food or meat that does not fall within the definition of ‘halal’ will then be considered as ‘non-halal. The definition of halal is further provided in https://www.smiic.org/en/project/24 (Organisation of Islamic Cooperation (OIC)/Standards and Metrology Institute for the Islamic Countries (SMIIC), OIC/SMIIC 1:2019 General Requirements for Halal Food. accessed on 8 Jun. 2022)

The term “test” used in conjunction with the term subject and/or animal in the present disclosure refers to an entity that is subjected to the method according to any aspect of the present invention and is the basis for an analysis application of the present invention. An “(individual) test subject”, an “(individual) group of test subjects” or a “test profile” or an ‘test animal derived product’ is therefore a (individual) subject or group of subjects being tested according to the invention or a profile being obtained or generated in this context. Conversely, the term “reference” shall denote, mostly predetermined, entities which are used for a comparison with the test entity. For example, the term ‘reference animal’ refers to an animal used for comparison or as a control in reference to the ‘test animal’. Similarly, the term ‘sample’ and/or ‘test animal-derived product sample’ used in accordance with any aspect of the present invention refers to an entity that may be subject to the method of the present invention. In particular, a sample may be any (test) animal-derived product that may be subject to the method of the present invention to determine the distinct certification by first determining the DNA methylation profile and then comparing this test methylation profile with a control. Blockchain may also be used to make the information easily available for the consumer.

The method according to any aspect of the present invention may be used to identify an unknown sample (i.e. animal-derived product sample) based on DNA methylation patterns. These DNA methylation patterns may then be compared with reference DNA methylation patterns to trace the animal-derived product sample back to the slaughterhouse or farmhouse from which the sample originates and then determine whether the unknown sample corresponds to an animal which has been slaughtered according to Kosher, Halal, non-Kosher or non-Halal practices. In this way, a buyer or a consumer of meat can verify that meat being sold as or marketed as being Kosher or Halal is genuine.

As used herein, the term “comprising” is to be construed as encompassing both “including” and “consisting of”, both meanings being specifically intended, and hence individually disclosed aspects of the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

In context of the present invention, the terms “methylation profile”, “methylation pattern”, “methylation state” or “methylation status,” are used herein to describe the state, situation or condition of methylation of a genomic sequence, and such terms refer to the characteristics of a DNA segment at a particular genomic locus in relation to methylation. Such characteristics include, but are not limited to, whether any of the cytosine (C) residues within this DNA sequence are methylated, location of methylated C residue(s), percentage of methylated C at any particular stretch of residues, and allelic differences in methylation due to, e.g., difference in the origin of the alleles.

The term “methylation status” refers to the status of a specific methylation site (i.e. methylated vs. non-methylated) which means a residue or methylation site is methylated or not methylated. Then, based on the methylation status of one or more methylation sites, a methylation profile may be determined. Accordingly, the term “methylation profile” or also “methylation pattern” refers to the relative or absolute concentration of methylated C residues or unmethylated C residues at any particular stretch of residues in the genomic material of a biological sample. For example, if cytosine (C) residue(s) not typically methylated within a DNA sequence are methylated, it may be referred to as “hypermethylated”; whereas if cytosine (C) residue(s) typically methylated within a DNA sequence are not methylated, it may be referred to as “hypomethylated”. Likewise, if the cytosine (C) residue(s) within a DNA sequence (e.g., the DNA from a sample nucleic acid from a test subject) are methylated as compared to another sequence from a different region or from a different individual (e.g., relative to normal nucleic acid or to the standard nucleic acid of the reference sequence), that sequence is considered hypermethylated compared to the other sequence. Alternatively, if the cytosine (C) residue(s) within a DNA sequence are not methylated as compared to another sequence from a different region or from a different individual, that sequence is considered hypomethylated compared to the other sequence. These sequences are said to be “differentially methylated”. Measurement of the levels of differential methylation may be done by a variety of ways known to those skilled in the art. One method is to measure the methylation level of individual interrogated CpG sites determined by the bisulfite sequencing method, as a non-limiting example.

The term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample. In particular, control refers to an animal derived product not slaughtered by a single cut across the throat severing both carotid arteries, both jugular veins, both vagus nerves, trachea and/or oesophagus and/or not having been bled to death.

The term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample. In particular, control refers to an animal derived product not slaughtered by a single cut across the throat severing both carotid arteries, both jugular veins, both vagus nerves, trachea and/or oesophagus and/or not having been bled to death.

As used herein, a “methylated nucleotide” or a “methylated nucleotide base” refers to the presence of a methyl moiety on a nucleotide base, where the methyl moiety is usually not present in a recognized typical nucleotide base. For example, cytosine in its usual form does not contain a methyl moiety on its pyrimidine ring, but 5-methylcytosine contains a methyl moiety at position 5 of its pyrimidine ring. Therefore, cytosine in its usual form may not be considered a methylated nucleotide and 5-methylcytosine may be considered a methylated nucleotide. In another example, thymine may contain a methyl moiety at position 5 of its pyrimidine ring, however, for purposes herein, thymine may not be considered a methylated nucleotide when present in DNA. Typical nucleotide bases for DNA are thymine, adenine, cytosine and guanine. Typical bases for RNA are uracil, adenine, cytosine and guanine. Correspondingly a “methylation site” is the location in the target gene nucleic acid region where methylation has the possibility of occurring. For example, a location containing CpG is a methylation site wherein the cytosine may or may not be methylated. In particular, the term “methylated nucleotide” refers to nucleotides that carry a methyl group attached to a position of a nucleotide that is accessible for methylation. These methylated nucleotides are usually found in nature and to date, methylated cytosine that occurs mostly in the context of the dinucleotide CpG, but also in the context of CpNpG- and CpNpN-sequences may be considered the most common. In principle, other naturally occurring nucleotides may also be methylated but they will not be taken into consideration with regard to any aspect of the present invention.

As used herein, a “CpG site” or “methylation site” is a nucleotide within a nucleic acid (DNA or RNA) that is susceptible to methylation either by natural occurring events in vivo or by an event instituted to chemically methylate the nucleotide in vitro.

As used herein, a “methylated nucleic acid molecule” refers to a nucleic acid molecule that contains one or more nucleotides that is/are methylated.

The term ‘epigenetic change’ as used herein refers to a chemical (e.g., methylation) change or protein (e.g., histones) change that takes place to a gene body or a promoter thereof. Through epigenetic changes, environmental factors like. diet, stress and prenatal nutrition can make an imprint on genes passed from one generation to the next.

The term “bisulfite” as used herein encompasses any suitable type of bisulfite, such as sodium bisulfite, or another chemical agent that is capable of chemically converting a cytosine (C) to a uracil (U) without chemically modifying a methylated cytosine and therefore can be used to differentially modify a DNA sequence based on the methylation status of the DNA, e.g., U.S. Pat. Pub. US 2010/0112595 (Menchen et al.). As used herein, a reagent that “differentially modifies” methylated or non-methylated DNA encompasses any reagent that modifies methylated and/or unmethylated DNA in a process through which distinguishable products result from methylated and non-methylated DNA, thereby allowing the identification of the DNA methylation status. Such processes may include, but are not limited to, chemical reactions (such as a C to U conversion by bisulfite) and enzymatic treatment (such as cleavage by a methylation-dependent endonuclease). Thus, an enzyme that preferentially cleaves or digests methylated DNA is one capable of cleaving or digesting a DNA molecule at a much higher efficiency when the DNA is methylated, whereas an enzyme that preferentially cleaves or digests unmethylated DNA exhibits a significantly higher efficiency when the DNA is not methylated.

In context of the present invention also any “non-bisulfite-based method” and “non-bisulfite-based quantitative method” are comprised to test for a methylation status at any given methylation site to be tested. Such terms refer to any method for quantifying methylated or non-methylated nucleic acid that does not require the use of bisulfite. The terms also refer to methods for preparing a nucleic acid to be quantified that do not require bisulfite treatment. Examples of non-bisulfite-based methods include, but are not limited to, methods for digesting nucleic acid using one or more methylation sensitive enzymes and methods for separating nucleic acid using agents that bind nucleic acid based on methylation status. The terms “methyl-sensitive enzymes” and “methylation sensitive restriction enzymes” are DNA restriction endonucleases that are dependent on the methylation state of their DNA recognition site for activity. For example, there are methyl-sensitive enzymes that cleave or digest at their DNA recognition sequence only if it is not methylated. Thus, an unmethylated DNA sample will be cut into smaller fragments than a methylated DNA sample. Similarly, a hypermethylated DNA sample will not be cleaved. In contrast, there are methyl-sensitive enzymes that cleave at their DNA recognition sequence only if it is methylated. As used herein, the terms “cleave”, “cut” and “digest” are used interchangeably.

A “biological sample” in context of the invention may comprise any biological material obtained from the subject or group of subjects that contains genomic material, and may be liquid, solid or both, may be tissue or bone, or a body fluid such as blood, lymph, etc. In particular, the biological sample useful for the present invention may comprise biological cells or fragments thereof. As used herein, the term “pre-selected methylation sites” refers to methylation sites that were selected from genes or regions that showed the highest degree of methylation variation during the training of the method and fulfils certain quality criteria such as a minimum sequencing coverage of ≥5× were considered and for ≥5 qualified CpG sites. Additionally, genes that have an average methylation level <0.1 or an average methylation level ≥0.9 can be excluded due to their limited dynamic range. “Reference methylation profiles” may be defined on the basis of multiple training samples using multivariate statistical methods, such as such as Principal Component analysis or Multi-Dimensional Scaling. In particular, the CpG site according to any aspect of the present invention, particularly in the pre-selected methylation sites is selected from the Table A below:

TABLE A
Relevant CpG sites that are differentially methylated

| No | chrom | position |
|---|---|---|
| 1 | 2 | 34511190 |
| 2 | 2 | 57572893 |
| 3 | 2 | 20693997 |
| 4 | Z | 17123376 |
| 5 | Z | 115673685 |
| 6 | Z | 24913614 |
| 7 | 4 | 58554384 |
| 8 | 18 | 8977183 |
| 9 | 4 | 45296016 |
| 10 | 7 | 18690074 |
| 11 | 2 | 123206194 |
| 12 | 1 | 49770596 |
| 13 | 5 | 29057117 |
| 14 | 21 | 2688710 |
| 15 | 1 | 47926496 |
| 16 | 13 | 11458665 |
| 17 | 5 | 19158349 |
| 18 | 11 | 18172296 |
| 19 | 10 | 12400953 |
| 20 | 19 | 8867093 |
| 21 | 4 | 39537311 |
| 22 | 13 | 6729058 |
| 23 | 10 | 11406434 |
| 24 | 4 | 21041627 |
| 25 | 26 | 5003532 |
| 26 | 8 | 16729492 |
| 27 | 4 | 39536841 |
| 28 | 7 | 24170000 |
| 29 | 2 | 23153225 |
| 30 | 1 | 130973501 |
| 31 | 19 | 8481362 |
| 32 | 1 | 14382900 |
| 33 | 10 | 6806714 |
| 34 | 10 | 12500816 |
| 35 | Z | 7668187 |
| 36 | 1 | 48250341 |
| 37 | 26 | 1544666 |
| 38 | 3 | 26860812 |
| 39 | 3 | 40601306 |
| 40 | 9 | 7624289 |
| 41 | 7 | 5077349 |
| 42 | 11 | 18808803 |
| 43 | 26 | 2590932 |
| 44 | 9 | 22225808 |
| 45 | 2 | 78548181 |
| 46 | 12 | 6363727 |
| 47 | 3 | 107890733 |
| 48 | 3 | 27258633 |
| 49 | 9 | 5216234 |
| 50 | 20 | 5579795 |
| 51 | 27 | 3637822 |
| 52 | 19 | 2411046 |
| 53 | 17 | 6189674 |
| 54 | 3 | 105678589 |
| 55 | 1 | 113283028 |
| 56 | 11 | 1459001 |
| 57 | 7 | 35189129 |
| 58 | 4 | 32571055 |
| 59 | 28 | 3800027 |
| 60 | 11 | 18808917 |
| 61 | 1 | 168855019 |
| 62 | 3 | 76755036 |
| 63 | 1 | 34377737 |
| 64 | 3 | 76739615 |
| 65 | 1 | 56825511 |
| 66 | 1 | 108957860 |
| 67 | 25 | 1876704 |
| 68 | 1 | 130278359 |
| 69 | 17 | 7904953 |
| 70 | 3 | 4193791 |
| 71 | 3 | 96461436 |
| 72 | 2 | 5096922 |
| 73 | 12 | 15606143 |
| 74 | 2 | 20877142 |
| 75 | 12 | 710959 |
| 76 | Z | 27177202 |
| 77 | 6 | 28147880 |
| 78 | 25 | 1107409 |
| 79 | 2 | 115921454 |
| 80 | 15 | 7347328 |
| 81 | 2 | 4980847 |
| 82 | 10 | 15213509 |
| 83 | 2 | 127761601 |
| 84 | 2 | 15482005 |
| 85 | 24 | 3786074 |
| 86 | 12 | 4380982 |
| 87 | 21 | 2332328 |
| 88 | Z | 11287812 |
| 89 | 1 | 36508947 |
| 90 | 7 | 301604 |
| 91 | 20 | 9983514 |
| 92 | 5 | 10355756 |
| 93 | 9 | 10672938 |
| 94 | 20 | 4533305 |
| 95 | 21 | 2332272 |
| 96 | 5 | 53496100 |
| 97 | 9 | 22079838 |
| 98 | 2 | 49110506 |
| 99 | 10 | 3477178 |
| 100 | 1 | 165873023 |
| 101 | Z | 16103001 |
| 102 | 5 | 27885744 |
| 103 | 2 | 4575039 |
| 104 | 33 | 431970 |
| 105 | 14 | 6596177 |
| 106 | 7 | 301591 |
| 107 | Z | 72829308 |
| 108 | 51 | 43891403 |
| 109 | 10 | 18678519 |
| 110 | 3 | 38237516 |
| 111 | 19 | 5832481 |
| 112 | 1 | 50837347 |
| 113 | Z | 33678917 |
| 114 | 4 | 89259160 |
| 115 | 19 | 5767147 |
| 116 | 4 | 40113483 |
| 117 | 5 | 14162871 |
| 118 | 10 | 10895845 |
| 119 | 20 | 1000718 |
| 120 | 1 | 194401733 |
| 121 | 3 | 58874863 |
| 122 | 20 | 10511507 |
| 123 | Z | 81360739 |
| 124 | 15 | 8003087 |
| 125 | 6 | 17322989 |
| 126 | 3 | 104905440 |
| 127 | 19 | 6022617 |
| 128 | 9 | 15414814 |
| 129 | 8 | 16295920 |
| 130 | 3 | 32261769 |
| 131 | 2 | 62413006 |
| 132 | 2 | 118636996 |
| 133 | 21 | 927717 |
| 134 | 25 | 1946574 |
| 135 | 31 | 22702842 |
| 136 | 5 | 12488676 |
| 137 | 1 | 169374378 |
| 138 | 1 | 35104436 |
| 139 | 28 | 153426 |
| 140 | 15 | 7003924 |
| 141 | 14 | 2268610 |
| 142 | 3 | 105678613 |
| 143 | 1 | 144628599 |
| 144 | 9 | 14072787 |
| 145 | 19 | 5363084 |
| 146 | 17 | 6765666 |
| 147 | 5 | 48084582 |
| 148 | 21 | 5073396 |
| 149 | 20 | 533436 |
| 150 | 11 | 10452379 |
| 151 | 4 | 49890681 |
| 152 | 5 | 52943641 |
| 153 | 2 | 46089114 |
| 154 | 23 | 1870014 |
| 155 | 2 | 41841626 |
| 156 | 24 | 4885733 |
| 157 | 4 | 6740626 |
| 158 | 3 | 76507235 |
| 159 | 5 | 58997382 |
| 160 | 17 | 8203614 |
| 161 | 27 | 3464008 |
| 162 | 11 | 841570 |
| 163 | 4 | 43608241 |
| 164 | 7 | 22569247 |
| 165 | 2 | 78548594 |
| 166 | 5 | 28115369 |
| 167 | 1 | 84532408 |
| 168 | 6 | 12121146 |
| 169 | 12 | 9675444 |
| 170 | 6 | 32236852 |
| 171 | 10 | 12734127 |
| 172 | Z | 11287708 |
| 173 | 8 | 16370376 |
| 174 | 19 | 1401235 |
| 175 | 1 | 144796538 |
| 176 | 2 | 102562607 |
| 177 | 8 | 7548136 |
| 178 | 14 | 5000387 |
| 179 | 1 | 56606169 |
| 180 | 9 | 9539267 |
| 181 | 18 | 3700175 |
| 182 | 1 | 61708721 |
| 183 | 8 | 29363057 |
| 184 | 6 | 11517878 |
| 185 | 4 | 88992493 |
| 186 | 4 | 10802755 |
| 187 | 3 | 12755149 |
| 188 | 2 | 21041245 |
| 189 | 14 | 957744 |
| 190 | Z | 27177213 |
| 191 | 7 | 28179710 |
| 192 | 4 | 11719574 |
| 193 | 5 | 24369896 |
| 194 | 19 | 5384384 |
| 195 | 24 | 4459949 |
| 196 | 6 | 32533539 |
| 197 | 21 | 4836900 |
| 198 | 1 | 16473870 |
| 199 | 1 | 110880942 |
| 200 | 8 | 28653132 |
| 201 | 28 | 2443299 |
| 202 | Z | 16048197 |
| 203 | 19 | 4088736 |
| 204 | 3 | 12789564 |
| 205 | 6 | 11221033 |
| 206 | 2 | 21010987 |
| 207 | 9 | 10619341 |
| 208 | 9 | 7925956 |
| 209 | 6 | 1549721 |
| 210 | 5 | 23458500 |
| 211 | 28 | 1477408 |
| 212 | 19 | 50552 |
| 213 | 26 | 3246923 |
| 214 | 8 | 6089739 |
| 215 | 1 | 3700593 |
| 216 | 13 | 16065472 |
| 217 | 4 | 53533077 |
| 218 | 5 | 52943689 |
| 219 | 27 | 1541894 |
| 220 | 26 | 4856960 |
| 221 | 12 | 2372012 |
| 222 | 1 | 110237976 |
| 223 | 1 | 84409963 |
| 224 | 1 | 39877658 |
| 225 | 5 | 11621859 |
| 226 | 25 | 976468 |
| 227 | 4 | 8695640 |
| 228 | 1 | 80519994 |
| 229 | 19 | 185280 |
| 230 | 3 | 48423158 |
| 231 | 1 | 39858634 |
| 232 | 8 | 16052167 |
| 233 | 1 | 91422737 |
| 234 | 18 | 3777721 |
| 235 | 12 | 7012484 |
| 236 | 1 | 77229485 |
| 237 | 3 | 49813062 |
| 238 | Z | 27270228 |
| 239 | 1 | 123415519 |
| 240 | 5 | 51772714 |
| 241 | 3 | 36638958 |
| 242 | 7 | 15096160 |
| 243 | 2 | 121482517 |
| 244 | 7 | 17289720 |
| 245 | NT_464303v1 | 5736 |
| 246 | 7 | 9938317 |
| 247 | 17 | 10895495 |
| 248 | 7 | 29790478 |
| 249 | 2 | 125171980 |
| 250 | 9 | 9528198 |
| 251 | 7 | 16281087 |
| 252 | 2 | 1390782 |
| 253 | 26 | 1519097 |
| 254 | Z | 11047446 |
| 255 | NT_464199v1 | 28427 |
| 256 | 3 | 108708555 |
| 257 | 4 | 5202992 |
| 258 | 3 | 19170252 |
| 259 | 17 | 10895665 |
| 260 | 7 | 6603676 |
| 261 | 3 | 105215220 |
| 262 | 26 | 1651982 |
| 263 | 10 | 10824700 |
| 264 | 4 | 39012274 |
| 265 | 14 | 7327840 |
| 266 | 2 | 42356641 |
| 267 | 5 | 53918500 |
| 268 | 4 | 57532309 |
| 269 | 6 | 11915935 |
| 270 | 5 | 28318496 |
| 271 | 1 | 50488695 |
| 272 | 7 | 11358921 |
| 273 | 1 | 15441151 |
| 274 | 6 | 10819414 |
| 275 | 1 | 39877753 |
| 276 | 8 | 2118531 |
| 277 | 17 | 2935626 |
| 278 | 17 | 7463984 |
| 279 | 20 | 5394896 |
| 280 | 17 | 4082722 |
| 281 | 15 | 7571659 |
| 282 | 1 | 144796614 |
| 283 | 1 | 165873085 |
| 284 | 4 | 66670233 |
| 285 | 16 | 168962 |
| 286 | 17 | 7673395 |
| 287 | 17 | 5741333 |
| 288 | 15 | 6891797 |
| 289 | 18 | 2274676 |
| 290 | 14 | 6696623 |
| 291 | 5 | 11091055 |
| 292 | 12 | 10148968 |
| 293 | 6 | 12121122 |
| 294 | 1 | 49441925 |
| 295 | 12 | 1581083 |
| 296 | 6 | 10819506 |
| 297 | 19 | 176255 |
| 298 | 11 | 10950629 |
| 299 | 7 | 11248389 |
| 300 | 2 | 121381306 |
| 301 | 2 | 85904873 |
| 302 | 2 | 129933052 |
| 303 | 3 | 39148221 |
| 304 | 9 | 15812625 |
| 305 | 1 | 35484379 |
| 306 | 17 | 2360133 |
| 307 | 8 | 1401347 |
| 308 | 2 | 66497871 |
| 309 | 15 | 5027241 |
| 310 | 12 | 7055474 |
| 311 | 2 | 3856589 |
| 312 | 1 | 193821130 |
| 313 | 13 | 18308448 |
| 314 | 1 | 68455273 |
| 315 | 2 | 114090502 |
| 316 | 4 | 60369319 |
| 317 | 27 | 4720035 |
| 318 | 10 | 967899 |
| 319 | 4 | 1985540 |
| 320 | 14 | 5059398 |
| 321 | 1 | 175460817 |
| 322 | 3 | 33946497 |
| 323 | 12 | 2259754 |
| 324 | 3 | 12583058 |
| 325 | 24 | 804341 |
| 326 | Z | 27444073 |
| 327 | 3 | 104543849 |
| 328 | 1 | 53092288 |
| 329 | 1 | 14903055 |
| 330 | 1 | 122962624 |
| 331 | 3 | 76739886 |
| 332 | 8 | 11915537 |
| 333 | 8 | 16296168 |
| 334 | 9 | 7724217 |
| 335 | 9 | 4626749 |
| 336 | 4 | 60368609 |
| 337 | Z | 37529420 |
| 338 | 13 | 3283054 |
| 339 | 1 | 194236534 |
| 340 | 18 | 10185868 |
| 341 | 18 | 1708416 |
| 342 | 8 | 16374372 |
| 343 | 8 | 14713737 |
| 344 | 8 | 16589375 |
| 345 | 9 | 5089285 |
| 346 | 9 | 8479123 |
| 347 | 3 | 8952808 |
| 348 | 1 | 137293780 |
| 349 | 13 | 13028604 |
| 350 | 5 | 53477916 |
| 351 | 8 | 28086175 |
| 352 | 2 | 26792587 |
| 353 | 12 | 1581381 |
| 354 | 5 | 39173222 |
| 355 | 4 | 86456090 |
| 356 | 5 | 38166099 |
| 357 | 3 | 46066858 |
| 358 | 20 | 5519899 |
| 359 | 11 | 6629490 |
| 360 | 18 | 9435327 |
| 361 | 3 | 102971530 |
| 362 | 19 | 4492763 |
| 363 | 23 | 4152128 |
| 364 | Z | 63907320 |
| 365 | 10 | 4390201 |
| 366 | 15 | 9763222 |
| 367 | 5_NT_460419v1 | 6746 |
| 368 | 19 | 199882 |
| 369 | 1 | 195334826 |
| 370 | 3 | 32362994 |
| 371 | NT_465658v1 | 7191 |
| 372 | 23 | 2381593 |
| 373 | 19 | 7203329 |
| 374 | Z | 65981131 |
| 375 | 5 | 13107975 |
| 376 | 2 | 57948024 |
| 377 | 1 | 56521314 |
| 378 | Z | 34328034 |
| 379 | 4 | 34288785 |
| 380 | 3 | 30399012 |
| 381 | 18 | 3700112 |
| 382 | 13 | 12290255 |
| 383 | 7 | 35847537 |
| 384 | 20 | 8145679 |
| 385 | NT_471028v1 | 3312 |
| 386 | 19 | 580436 |
| 387 | 5 | 9279301 |
| 388 | 23 | 3739407 |
| 389 | 24 | 375697 |
| 390 | 1 | 79199586 |
| 391 | 17 | 8205987 |
| 392 | 31 | 26344201 |
| 393 | 18 | 7373876 |
| 394 | 3 | 67720493 |
| 395 | 2 | 129925091 |
| 396 | 4 | 2123054 |
| 397 | 20 | 10287258 |
| 398 | 6 | 24169226 |
| 399 | 5 | 48066943 |
| 400 | 26 | 3571402 |
| 401 | 3 | 7315133 |
| 402 | 7 | 2239688 |
| 403 | 17 | 8377056 |
| 404 | 28 | 153360 |
| 405 | 3 | 2421530 |
| 406 | 4 | 39771776 |
| 407 | 6 | 9407093 |
| 408 | 17 | 5799733 |
| 409 | 4 | 45296889 |
| 410 | 28 | 3642946 |
| 411 | 2 | 13471116 |
| 412 | 3 | 31394938 |
| 413 | 6 | 22239587 |
| 414 | 8 | 25610078 |
| 415 | 1 | 153736291 |
| 416 | 8 | 6618970 |
| 417 | 10 | 2333800 |
| 418 | 14 | 12811447 |
| 419 | 7 | 22755583 |
| 420 | 6 | 27262610 |
| 421 | 27 | 4719697 |
| 422 | 11 | 10256374 |
| 423 | 11 | 11215844 |
| 424 | 3 | 12755221 |
| 425 | 2 | 121422611 |
| 426 | 9 | 10594523 |
| 427 | 2 | 88162809 |
| 428 | 5 | 9737337 |
| 429 | 17 | 1980788 |
| 430 | 1 | 24855132 |
| 431 | 1 | 71113923 |
| 432 | 11 | 10194539 |
| 433 | 1 | 76766539 |
| 434 | NT_464142v1 | 64936 |
| 435 | 3 | 38336190 |
| 436 | 17 | 8376829 |
| 437 | 15 | 5989722 |
| 438 | 7 | 15587386 |
| 439 | 3 | 21854776 |
| 440 | 11 | 6629712 |
| 441 | 1 | 178990900 |
| 442 | 12 | 10134215 |
| 443 | 8 | 13384214 |
| 444 | 1 | 12877821 |
| 445 | 9 | 6855496 |
| 446 | 1 | 175461006 |
| 447 | 3 | 105678619 |
| 448 | 2 | 130013087 |
| 449 | 14 | 8217810 |
| 450 | 14 | 10043625 |
| 451 | 17 | 9978114 |
| 452 | 2 | 42913387 |
| 453 | 19 | 5832942 |
| 454 | 15 | 5823857 |
| 455 | 2 | 46089139 |
| 456 | 1 | 188916642 |
| 457 | 3 | 46372522 |
| 458 | 1 | 175460839 |
| 459 | 14 | 5261997 |
| 460 | 12 | 11951638 |
| 461 | 4 | 88992878 |
| 462 | 3 | 51285929 |
| 463 | 1 | 65578611 |
| 464 | 11 | 10578956 |
| 465 | 13 | 13224232 |
| 466 | 5 | 39113705 |
| 467 | 21 | 1483434 |
| 468 | 18 | 3699658 |
| 469 | 9 | 4676234 |
| 470 | 1 | 169234551 |
| 471 | 1 | 44860990 |
| 472 | 10 | 2315851 |
| 473 | 13 | 17037566 |
| 474 | 1 | 50703527 |
| 475 | 9 | 4850885 |
| 476 | 1 | 83226834 |
| 477 | 1 | 65578655 |
| 478 | 4 | 73695235 |
| 479 | 14 | 6926191 |
| 480 | 2 | 111960400 |
| 481 | 1 | 153739829 |
| 482 | 1 | 53953094 |
| 483 | 7 | 22245891 |
| 484 | 1 | 88411659 |
| 485 | 1 | 51088644 |
| 486 | 1 | 92330716 |
| 487 | 1 | 65578725 |
| 488 | 3 | 77142645 |
| 489 | 2 | 91651196 |
| 490 | 1 | 76766536 |
| 491 | 7 | 28205381 |
| 492 | 4 | 46058642 |
| 493 | 2 | 124732645 |
| 494 | 7 | 9697869 |
| 495 | 3 | 65294828 |
| 496 | 1 | 26767903 |
| 497 | 2 | 93915362 |
| 498 | 1 | 88759238 |
| 499 | 1 | 92271976 |
| 500 | 5 | 480120 |
| 501 | NT_468071v1 | 11202 |
| 502 | 10 | 10680859 |
| 503 | 1 | 175806942 |
| 504 | 1 | 7556524 |
| 505 | 1 | 54593907 |
| 506 | 1 | 44871524 |
| 507 | 1 | 56825448 |
| 508 | 1 | 79310370 |
| 509 | 10 | 2277642 |
| 510 | 10 | 16470610 |
| 511 | 13 | 13567090 |
| 512 | 14 | 8154906 |
| 513 | 17 | 10279957 |
| 514 | 19 | 934610 |
| 515 | 3 | 26827400 |
| 516 | 3 | 49135422 |
| 517 | 4 | 33043830 |
| 518 | 8 | 2489495 |
| 519 | 8 | 5304565 |
| 520 | Z | 10464173 |
| 521 | 3 | 33946905 |
| 522 | 3 | 33980543 |
| 523 | 3 | 35737558 |
| 524 | 3 | 37962586 |
| 525 | 3 | 39178569 |
| 526 | 3 | 42000296 |
| 527 | 3 | 46066726 |
| 528 | 3 | 48325731 |
| 529 | 3 | 48792278 |
| 530 | 3 | 49036683 |
| 531 | 3 | 50934801 |
| 532 | 3 | 52022798 |
| 533 | 3 | 57998308 |
| 534 | 3 | 60009081 |
| 535 | 3 | 62286891 |
| 536 | 3 | 72999617 |
| 537 | 3 | 75813980 |
| 538 | 3 | 77142698 |
| 539 | 3 | 77740997 |
| 540 | 3 | 77741067 |
| 541 | 3 | 77806851 |
| 542 | 3 | 79869584 |
| 543 | 3 | 82094768 |
| 544 | 3 | 87049472 |
| 545 | 3 | 104543506 |
| 546 | 33 | 431964 |
| 547 | 3_NT463456v1 | 37759 |
| 548 | 4 | 2503560 |
| 549 | 4 | 3023101 |
| 550 | 4 | 4377067 |
| 551 | 4 | 5154481 |
| 552 | 4 | 8581737 |
| 553 | 4 | 8734750 |
| 554 | 4 | 10712066 |
| 555 | 4 | 11020158 |
| 556 | 4 | 12409848 |
| 557 | 4 | 13440210 |
| 558 | 4 | 15354497 |
| 559 | 4 | 21510615 |
| 560 | 4 | 21597219 |
| 561 | 4 | 29981474 |
| 562 | 4 | 29981612 |
| 563 | 4 | 30237031 |
| 564 | 4 | 31634271 |
| 565 | 4 | 31775781 |
| 566 | 4 | 31797849 |
| 567 | 4 | 31798007 |
| 568 | 4 | 32235697 |
| 569 | 4 | 34147184 |
| 570 | 4 | 34634614 |
| 571 | 4 | 45304161 |
| 572 | 4 | 47198051 |
| 573 | 4 | 50421355 |
| 574 | 23 | 446710 |
| 575 | 23 | 1897115 |
| 576 | 23 | 4988905 |
| 577 | 24 | 2183046 |
| 578 | 24 | 2745230 |
| 579 | 25 | 976498 |
| 580 | 25 | 1102452 |
| 581 | 25 | 1922074 |
| 582 | 25 | 1946155 |
| 583 | 25 | 2053338 |
| 584 | 25 | 2379274 |
| 585 | 26 | 2591052 |
| 586 | 27 | 1273378 |
| 587 | 27 | 1528397 |
| 588 | 27 | 1541617 |
| 589 | 27 | 1609933 |
| 590 | 27 | 3878658 |
| 591 | 27 | 4209484 |
| 592 | 28 | 700769 |
| 593 | 28 | 785678 |
| 594 | 28 | 799393 |
| 595 | 28 | 2802446 |
| 596 | 28 | 4072263 |
| 597 | 3 | 12990210 |
| 598 | 3 | 19773897 |
| 599 | 3 | 24008503 |
| 600 | 3 | 26263279 |
| 601 | 3 | 26264001 |
| 602 | 3 | 26757632 |
| 603 | 3 | 26827383 |
| 604 | 3 | 31006184 |
| 605 | 3 | 31169966 |
| 606 | 3 | 31416605 |
| 607 | 2 | 50003872 |
| 608 | 2 | 60065457 |
| 609 | 2 | 60969061 |
| 610 | 2 | 62413026 |
| 611 | 2 | 66497875 |
| 612 | 2 | 75844300 |
| 613 | 2 | 79703088 |
| 614 | 2 | 90554149 |
| 615 | 2 | 100917787 |
| 616 | 2 | 101292338 |
| 617 | 2 | 102813119 |
| 618 | 2 | 103329886 |
| 619 | 2 | 106422461 |
| 620 | 2 | 108042776 |
| 621 | 2 | 108043217 |
| 622 | 2 | 108413855 |
| 623 | 2 | 108413888 |
| 624 | 2 | 109563147 |
| 625 | 2 | 116530961 |
| 626 | 2 | 116531160 |
| 627 | 2 | 123333539 |
| 628 | 2 | 123333776 |
| 629 | 2 | 123623354 |
| 630 | 2 | 128974344 |
| 631 | 2 | 129932927 |
| 632 | 2 | 132629535 |
| 633 | 2 | 139673759 |
| 634 | 2 | 146178598 |
| 635 | 20 | 8162867 |
| 636 | 20 | 10275863 |
| 637 | 20 | 12592864 |
| 638 | 21 | 1726961 |
| 639 | 21 | 1824168 |
| 640 | 21 | 2332382 |
| 641 | 18 | 10470282 |
| 642 | 19 | 1792458 |
| 643 | 19 | 3779599 |
| 644 | 19 | 5325089 |
| 645 | 19 | 7633138 |
| 646 | 19 | 7633191 |
| 647 | 19 | 8867086 |
| 648 | 2 | 4915125 |
| 649 | 2 | 6233872 |
| 650 | 2 | 6287263 |
| 651 | 2 | 12592185 |
| 652 | 2 | 12854026 |
| 653 | 2 | 15015618 |
| 654 | 2 | 19106544 |
| 655 | 2 | 19555468 |
| 656 | 2 | 20654008 |
| 657 | 2 | 20877171 |
| 658 | 2 | 21020630 |
| 659 | 2 | 21776645 |
| 660 | 2 | 23152655 |
| 661 | 2 | 30653036 |
| 662 | 2 | 30964872 |
| 663 | 2 | 31517731 |
| 664 | 2 | 34511511 |
| 665 | 2 | 36807563 |
| 666 | 2 | 44148776 |
| 667 | 2 | 45606950 |
| 668 | 2 | 47274383 |
| 669 | 9 | 1125703 |
| 670 | 9 | 1432683 |
| 671 | 9 | 1621719 |
| 672 | 9 | 4819197 |
| 673 | 9 | 5216686 |
| 674 | 9 | 5273930 |
| 675 | 9 | 7969508 |
| 676 | 9 | 8025607 |
| 677 | 9 | 8479246 |
| 678 | 9 | 9622397 |
| 679 | 9 | 10672930 |
| 680 | 9 | 12209165 |
| 681 | 9 | 15449427 |
| 682 | 9 | 15664766 |
| 683 | 9 | 20353960 |
| 684 | 9 | 22079462 |
| 685 | 9 | 22364985 |
| 686 | NT_466341v1 | 10512 |
| 687 | Z | 2265489 |
| 688 | Z | 17067814 |
| 689 | Z | 24036072 |
| 690 | Z | 24036312 |
| 691 | Z | 24913899 |
| 692 | Z | 27815215 |
| 693 | Z | 31979924 |
| 694 | Z | 35676605 |
| 695 | Z | 36792135 |
| 696 | Z | 40093320 |
| 697 | Z | 46063014 |
| 698 | Z | 50823119 |
| 699 | Z | 52080666 |
| 700 | Z | 52630840 |
| 701 | Z | 72177473 |
| 702 | 13 | 14499771 |
| 703 | 13 | 15760688 |
| 704 | 13 | 17043224 |
| 705 | 13 | 17043375 |
| 706 | 14 | 2330596 |
| 707 | 14 | 2610614 |
| 708 | 14 | 6595973 |
| 709 | 14 | 7315747 |
| 710 | 14 | 7451783 |
| 711 | 14 | 12900892 |
| 712 | 14 | 13057958 |
| 713 | 15 | 4517586 |
| 714 | 15 | 4987756 |
| 715 | 15 | 5106738 |
| 716 | 15 | 5986957 |
| 717 | 15 | 6018471 |
| 718 | 15 | 7042315 |
| 719 | 15 | 8003013 |
| 720 | 15 | 11346421 |
| 721 | 15 | 12108868 |
| 722 | 16 | 105715 |
| 723 | 17 | 1439118 |
| 724 | 17 | 2457632 |
| 725 | 17 | 2588441 |
| 726 | 17 | 3817120 |
| 727 | 17 | 4082619 |
| 728 | 17 | 6717101 |
| 729 | 17 | 8592228 |
| 730 | 17 | 9171080 |
| 731 | 17 | 10088808 |
| 732 | 18 | 7515126 |
| 733 | 18 | 7875787 |
| 734 | 18 | 9697090 |
| 735 | 1 | 166728921 |
| 736 | 1 | 183325914 |
| 737 | 1 | 185282396 |
| 738 | 1 | 188309833 |
| 739 | 1 | 194782826 |
| 740 | 1 | 195388710 |
| 741 | 1 | 196146823 |
| 742 | 10 | 968395 |
| 743 | 10 | 2096300 |
| 744 | 10 | 2277755 |
| 745 | 10 | 2505096 |
| 746 | 10 | 6959419 |
| 747 | 10 | 7035652 |
| 748 | 10 | 7767102 |
| 749 | 10 | 18093950 |
| 750 | 10 | 20006398 |
| 751 | 11 | 1329819 |
| 752 | 11 | 10194367 |
| 753 | 11 | 11467077 |
| 754 | 11 | 11492065 |
| 755 | 11 | 16009840 |
| 756 | 12 | 1444617 |
| 757 | 12 | 2401731 |
| 758 | 12 | 2575431 |
| 759 | 12 | 2911021 |
| 760 | 12 | 6759370 |
| 761 | 12 | 10178070 |
| 762 | 12 | 10809661 |
| 763 | 12 | 11952324 |
| 764 | 12 | 16343688 |
| 765 | 12 | 18132965 |
| 766 | 13 | 4123382 |
| 767 | 13 | 9484641 |
| 768 | 13 | 11458667 |
| 769 | 13 | 12045999 |
| 770 | 7 | 13949196 |
| 771 | 7 | 14319998 |
| 772 | 7 | 14832870 |
| 773 | 7 | 15906091 |
| 774 | 7 | 25408321 |
| 775 | 7 | 26827807 |
| 776 | 8 | 2271335 |
| 777 | 8 | 5110731 |
| 778 | 8 | 7548409 |
| 779 | 8 | 14361683 |
| 780 | 8 | 15791045 |
| 781 | 8 | 16370427 |
| 782 | 8 | 19012408 |
| 783 | 8 | 19105674 |
| 784 | 8 | 20844260 |
| 785 | 8 | 20862635 |
| 786 | 8 | 21330776 |
| 787 | 8 | 27993362 |
| 788 | 7 | 27306835 |
| 789 | 7 | 27538837 |
| 790 | 7 | 28565262 |
| 791 | 7 | 29084206 |
| 792 | 7 | 29525425 |
| 793 | 7 | 30383908 |
| 794 | 7 | 30593598 |
| 795 | 7 | 33455887 |
| 796 | 7 | 33793138 |
| 797 | 7 | 35330918 |
| 798 | 7 | 35330947 |
| 799 | 7 | 35848239 |
| 800 | 1 | 155080938 |
| 801 | 1 | 155581060 |
| 802 | 5 | 38034094 |
| 803 | 5 | 44495103 |
| 804 | 5 | 45270723 |
| 805 | 5 | 51814426 |
| 806 | 5 | 52015072 |
| 807 | 5 | 53299189 |
| 808 | 5 | 53390948 |
| 809 | 5 | 53566111 |
| 810 | 5 | 54521468 |
| 811 | 5 | 54656647 |
| 812 | 5 | 56343550 |
| 813 | 5 | 58703602 |
| 814 | 6 | 5064205 |
| 815 | 6 | 11151853 |
| 816 | 6 | 11188537 |
| 817 | 6 | 11582592 |
| 818 | 6 | 11761740 |
| 819 | 6 | 16194199 |
| 820 | 6 | 21978838 |
| 821 | 6 | 22083181 |
| 822 | 6 | 31431558 |
| 823 | 6 | 31473215 |
| 824 | 6 | 32181323 |
| 825 | 6 | 32533531 |
| 826 | 6 | 32568586 |
| 827 | 7 | 4813474 |
| 828 | 7 | 5038366 |
| 829 | 7 | 5060102 |
| 830 | 4 | 51454889 |
| 831 | 4 | 55109704 |
| 832 | 4 | 60528211 |
| 833 | 4 | 61707165 |
| 834 | 4 | 66670202 |
| 835 | 1 | 34681 |
| 836 | 1 | 524205 |
| 837 | 1 | 665546 |
| 838 | 1 | 959766 |
| 839 | 1 | 959793 |
| 840 | 1 | 1061369 |
| 841 | 1 | 20101427 |
| 842 | 1 | 20529755 |
| 843 | 1 | 22090541 |
| 844 | 1 | 22105964 |
| 845 | 1 | 23223035 |
| 846 | 1 | 24805622 |
| 847 | 1 | 24806572 |
| 848 | 1 | 28910262 |
| 849 | 1 | 33609212 |
| 850 | 1 | 42851095 |
| 851 | 1 | 44172676 |
| 852 | 1 | 46517622 |
| 853 | 1 | 47827684 |
| 854 | 1 | 47828170 |
| 855 | 1 | 47996727 |
| 856 | 1 | 49461091 |
| 857 | 1 | 49770584 |
| 858 | 1 | 49923743 |
| 859 | 1 | 50116959 |
| 860 | 1 | 54593904 |
| 861 | 1 | 56606098 |
| 862 | 1 | 56825555 |
| 863 | 1 | 61015958 |
| 864 | 1 | 61016067 |
| 865 | 1 | 78401374 |
| 866 | 1 | 78401723 |
| 867 | 1 | 79293224 |
| 868 | 1 | 80519649 |
| 869 | 1 | 84657280 |
| 870 | 1 | 84657286 |
| 871 | 1 | 87297037 |
| 872 | 1 | 87498913 |
| 873 | 1 | 88405899 |
| 874 | 1 | 89125947 |
| 875 | 1 | 102484686 |
| 876 | 1 | 105596806 |
| 877 | 1 | 105859382 |
| 878 | 1 | 110710757 |
| 879 | 1 | 111034338 |
| 880 | 1 | 118204632 |
| 881 | 1 | 124980421 |
| 882 | 1 | 129210612 |
| 883 | 1 | 130493042 |
| 884 | 1 | 130543594 |
| 885 | 1 | 131937808 |
| 886 | 1 | 133908500 |
| 887 | 4 | 77976506 |
| 888 | 4 | 78949903 |
| 889 | 4 | 83226239 |
| 890 | 4 | 85487431 |
| 891 | 4 | 88447258 |
| 892 | 4 | 88894627 |
| 893 | 4 | 88894964 |
| 894 | 4 | 89096143 |
| 895 | 4 | 89309689 |
| 896 | 5 | 10408062 |
| 897 | 5 | 11924701 |
| 898 | 5 | 12183678 |
| 899 | 5 | 13200139 |
| 900 | 5 | 14214536 |
| 901 | 5 | 14626826 |
| 902 | 5 | 14754310 |
| 903 | 5 | 15857395 |
| 904 | 5 | 16673990 |
| 905 | 5 | 18315526 |
| 906 | 5 | 19158441 |
| 907 | 5 | 19158446 |
| 908 | 5 | 19495119 |
| 909 | 5 | 22761531 |
| 910 | 5 | 25139165 |
| 911 | 5 | 26210040 |
| 912 | 5 | 26628736 |
| 913 | 5 | 27936505 |
| 914 | 5 | 28451085 |
| 915 | 5 | 29056520 |
| 916 | 5 | 34703798 |

The term “pre-determined reference profile” used in the context of the present invention refers to a typical or standard methylation profile of the genomic material of a type of reference animal-derived product that is confirmed to be correctly labelled or certified. In one example, the pre-determined reference profile may be used in the context of a control animal, where the control animal has been correctly certified (i.e. where the control animal has neither been slaughtered by a single cut across the throat severing both carotid arteries, both jugular veins, both vagus nerves, trachea and/or oesophagus nor of having been bled to death). The control animal may thus have been slaughtered using non-halal and non-kosher means of slaughtering that may be termed the normal means of slaughtering. A panel of pre-determined reference profiles for control animals may include profiles from different samples that have been obtained from different parts of control animals (not slaughtered by a single cut across the throat severing both carotid arteries, both jugular veins, both vagus nerves, trachea and/or oesophagus nor of having been bled to death). For example, the panel of pre-determined reference profiles may include at least one profile for egg, at least one profile for meat (muscle, tissue, organs etc.), at least one profile for milk and the like. Each of these samples may have its own unique pre-determined methylation reference profile that also forms a part of the panel of pre-determined reference profiles

In another example, the pre-determined reference profile may be used in the context of reference animals that may have been slaughtered by a single cut across the throat severing both carotid arteries, both jugular veins, both vagus nerves, trachea and/or oesophagus and/or having been bled to death. A panel of pre-determined reference profiles may be prepared for different samples that are from animals that have been confirmed to be slaughtered by a single cut across the throat severing both carotid arteries, both jugular veins, both vagus nerves, trachea and/or oesophagus and/or having been bled to death. Again here, there may be a panel of pre-determined reference profiles for each product that is derived from the animal that has been slaughtered by a single cut across the throat severing both carotid arteries, both jugular veins, both vagus nerves, trachea and/or oesophagus and/or having been bled to death. For example, the panel of pre-determined reference profiles may include at least one profile for egg, at least one profile for meat (muscle, tissue, organs etc.), at least one profile for milk and the like from animals that have been slaughtered by a single cut across the throat severing both carotid arteries, both jugular veins, both vagus nerves, trachea and/or oesophagus and/or having been bled to death. Each of these samples may have its own unique pre-determined methylation reference profile that also forms a part of the panel of pre-determined reference profiles

For example, the control may a type of meat of the same species or the same animal taxon as the animal-derived product sample or unknown sample which has a known certification. Alternatively, the pre-determined reference profile may be obtained from different types of meat with a known certification. In one example, the control may be chicken meat that is certified ‘halal’. The halal chicken meat may be a mix of different parts of the chicken (i.e. breast, thigh, kidney, liver etc.). In another example, the halal chicken meat may be from one part of a chicken. From the halal chicken meat, a methylation profile may be obtained to be the pre-determined reference profile (the control) that may be used to determine if the test sample (meat) is halal or non-halal by determining if the methylation profile of the test sample is significantly similar to the pre-determined reference profile. The methylation profile of different types of halal meat from one species of animal may be identical. The methylation profile of different types of halal meat from different species of animals may also be identical. In one example, the control may be chicken meat that is certified ‘kosher’. The kosher chicken meat may be a mix of different parts of the chicken (i.e. breast, thigh, kidney, liver etc.). In another example, the kosher chicken meat may be from one part of a chicken. From the kosher chicken meat, a methylation profile may be obtained to be the pre-determined reference profile (the control) that may be used to determine if the test sample (meat) is kosher or non-kosher by determining if the methylation profile of the test sample is significantly similar to the pre-determined reference profile. The methylation profile of different types of kosher meat from one species of animal may be identical. The methylation profile of different types of kosher meat from different species of animals may also be identical.

There may be a compilation of several pre-determined reference profiles and comparing the methylation profile of the test sample with the pre-determined reference profiles in the compilation may enable identifying the specific pre-determined reference profile that is (significantly) similar to the methylation profile of the test sample and then the test sample may be confirmed to be slaughtered by a single cut across the throat severing both carotid arteries, both jugular veins, both vagus nerves, trachea and/or oesophagus and/or having been bled to death or not. In one example, the pre-determined reference profiles may include methylation profiles of different parts of meat (i.e. breast, thigh, kidney, liver, shoulder, ribs, intestines, etc.) from an animal (chicken, goat, cow, lamb, sheep etc.) that has been slaughtered by a single cut across the throat severing both carotid arteries, both jugular veins, both vagus nerves, trachea and/or oesophagus and/or having been bled to death.

In particular, the panel of predetermined reference methylation profiles according to any aspect of the present invention is distinct for different test animal-derived products. That is to say, each predetermined reference methylation profile is distinct for a single animal-derived product. The panel of predetermined reference methylation profiles may thus include many different predetermined reference methylation profiles from different parts of an animal or several animals of the same biological taxon as the test animal. There will also be different panels of predetermined reference methylation profiles for different animal taxon, and the relevant panel of predetermined reference methylation profiles unique for an animal taxon will depend on the animal taxon of the test animal.

The term “significantly similar” in context of the present disclosure, and in particular in context with the comparison of methylation profiles (such as the comparison between test profiles (from test subject(s) (i.e. sample X) and reference profiles) shall mean a similarity observed by statistical means (i.e. by using bioinformatics) and/or also by observation using the eye. A significant similarity is observed for example if a test profile overlaps with a reference profile that is defined by multiple training samples through multivariate statistical methods, such as Principal Component analysis or Multi-Dimensional Scaling. In particular, a test profile is significantly similar to the pre-determined reference profile if more than 50, 55, 60, 65, 70, 75, 80, 85, 90, 95, 96, 97, 98, 99% % of the methylation pattern/profile overlaps with that of the reference profile. A similarity of a test profile to more than one, such as two, three or even all reference profile reduces the significance of the similarity. Of course, the similarity of the test methylation profile to the reference methylation profile takes into consideration the experimental error that occurs in all methods.

The animal-derived product sample may be a single type of meat, different types of meat, a single part of a type of meat, different parts of a single type of meat or different parts of different types of meat. The sample may be from any biological entity having a DNA genome and DNA genome methylation. In particular, the methylation site is a CpG site. The biological entity may be any animal excluding a pig. In particular, the animal may be selected from the group consisting of chicken, lamb, camel, cow, goat, sheep, horse, donkey, turkey, duck, goose, quail, rabbit and mule.

More in particular, the animal may be selected from the group consisting of cow, sheep, goat, camel, chicken, goose, duck and turkey. The term ‘meat’ herein may thus be understood to include chicken, lamb, beef, mutton, goat, camel, chicken, goose, duck, turkey and mixtures thereof.

The one or more pre-selected methylation sites in (a) are methylation sites associated with tissue specific gene expression, preferably wherein the pre-selected methylation sites are associated with gene expression of one distinct tissue.

The tissue may be selected from
(i) metabolic tissue such as gut tissue, said gut tissue preferably being ileum or jejunum,
(ii) muscular tissue,
(iii) skin tissue, and
(iv) organ tissue, said organ tissue preferably being hepatic and/or pancreatic tissue.

According to yet another aspect of the present invention, there is provided a method of verifying certification of a test animal-derived product sample that has been certified to derive from a test animal that was slaughtered by a single cut across the throat severing both carotid arteries, both jugular veins, both vagus nerves, trachea and/or oesophagus and/or that was bled to death, the method comprising the steps of:
(a) determining a test methylation profile of one or more pre-selected methylation sites within the genomic material obtained from the test animal derived product sample; and
(b) comparing the test methylation profile obtained from (a) with a reference methylation profile obtained from a control animal of the same biological taxon of the test animal from which the product sample is derived from, where the control animal is not slaughtered by a single cut across the throat severing both carotid arteries, both jugular veins, both vagus nerves, trachea and/or oesophagus and/or the control animal is not bled to death

wherein a difference in the test methylation profile of (a) compared to the reference methylation profile from the control animal, is indicative of the test animal having been slaughtered by a single cut across the throat severing both carotid arteries, both jugular veins, both vagus nerves, trachea and/or oesophagus and/or of the test animal having been bled to death and the test animal-derived product being correctly certified, and

wherein a similarity in the test methylation profile of (a) compared to the reference methylation profile of the control animal, is indicative of the test animal not having been slaughtered by a single cut across the throat severing both carotid arteries, both jugular veins, both vagus nerves, trachea and/or oesophagus and/or of the test animal having been bled to death and the test animal-derived product being wrongly certified, and

wherein the pre-selected methylation sites are CpG sites selected from genes or regions of genomic DNA from the control and test animal that shows the highest degree of methylation variation during the training of the method.

According to a further aspect of the present invention, there is provided a method for confirming or rejecting an assumed certification of an animal derived product sample, the method comprising
comparison of a test methylation profile obtained from genomic material of the animal derived product sample with a reference methylation profile obtained from a control animal of the same biological taxon of the test animal from which the product sample is derived from, where the control animal is not slaughtered by a single cut across the throat severing both carotid arteries, both jugular veins, both vagus nerves, trachea and/or oesophagus and/or the control animal is not bled to death, wherein a difference in the test methylation profile compared to the reference methylation profile from the control animal, is indicative of the test animal having been slaughtered by a single cut across the throat severing both carotid arteries, both jugular veins, both vagus nerves, trachea and/or oesophagus and/or of the test animal having been bled to death wherein the assumed certification is that the test animal derived product sample is kosher or halal where the test animal is slaughtered by a single cut across the throat severing both carotid arteries, both jugular veins, both vagus nerves, trachea and/or oesophagus and/or the test animal is bled to death.

According to another aspect of the present invention, there is provided a method of determining if a test animal-derived product sample, is obtained from a test animal that has been bled to death or slaughtered by a single cut across the throat severing both carotid arteries, both jugular veins, both vagus nerves, trachea and/or oesophagus the method comprising:
comparison of a test methylation profile obtained from genomic material of the test animal derived product sample with a reference methylation profile obtained from a control animal of the same biological taxon of the test animal from which the product sample is derived from, where the control animal is not slaughtered by a single cut across the throat severing both carotid arteries, both jugular veins, both vagus nerves, trachea and/or oesophagus and/or the control animal is not bled to death.

In particular, the certification is kosher or halal where the animal is slaughtered by a cut across the throat severing both carotid arteries, both jugular veins, both vagus nerves, the trachea and/or the oesophagus. Additionally, or alternatively, the certification kosher or halal where the animal is drained of blood.

According to yet a further aspect of the present invention, there is provided a certification of a test animal derived product sample certifying that the test animal was slaughtered by a single cut across the throat severing both carotid arteries, both jugular veins, both vagus nerves, the trachea and/or the oesophagus,
wherein the method for determining that the test animal was slaughtered by a single cut across the throat severing both carotid arteries, both jugular veins, both vagus nerves, the trachea and/or the oesophagus comprises the steps of:
   (a) determining a test methylation profile of one or more pre-selected methylation sites within the genomic material contained in the test animal-derived product sample; and
   (b) comparing the test methylation profile determined in (a) with a panel of predetermined reference methylation profiles of the same biological taxon of the test animal from which the product sample derives, wherein each of the predetermined reference methylation profiles is from a different reference animal and/or different supplier,

wherein if the test methylation profile of (a) is significantly similar to one of the predetermined reference methylation profiles, the test animal was slaughtered by a single cut across the throat severing both carotid arteries, both jugular veins, both vagus nerves, the trachea and/or the oesophagus.

Additionally, or alternatively, the test animal is drained of blood.

According to yet another aspect of the present invention, there is provided a use of DNA methylation profiling for determining for certifying of an animal-derived product sample. In particular, the certification of the animal-derived product sample is kosher or halal where the test animal is slaughtered by a single cut across the throat severing both carotid arteries, both jugular veins, both vagus nerves, trachea and/or oesophagus and/or the test animal is bled to death.

### BRIEF DESCRIPTIONS OF FIGURES

FIG. 1 is the results of Principle Component Analysis (PCA) of 6706643 CpG sites with min coverage 10 in all halal and non-halal samples.

FIG. 2 is the results of Principle Component Analysis (PCA) of 76339 Differentially methylated positions (DMPs) identified.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

### Wet-Lab Methodology

Halal and non halal chicken breast meat were purchased from five different vendors. The vendors for halal meat include Pasar, ZAC Butchery, Hego premium, RedMart and Keesong, whereas non halal meat was purchased from Pasar, Tegel, Farm fresh, Ryan and AW's in Singapore. Genomic DNA was purified from the breast tissue samples using DNeasy Blood & Tissue Kit (Qiagen) and is quantified using PicroGreen or NanoDrop™ 2000.

The genomic DNA (500 ng) from breast tissue samples was used to prepare libraries for Whole Genome Bisulfite Sequencing (WGBS). The sequencing of the libraries was performed by a third party on a NovaSeq platform which generated 125 GB data per sample with 20× coverage.

| Halal | Non-Halal |
|---|---|
| Brand | Brand |
| Pasar | Pasar |
| ZAC Butchery | Tegel |
| Hego premium | Farm fresh |
| RedMart | Ryan |
| Keesong | AW's |

### Computational Methodology

### Processing:

Sequenced reads were trimmed and mapped with BSMAP1 version 2.5 using the assembly version 5.0 of the chicken (Gallus gallus) genome as reference sequence. After deduplication using picard2 (http://broadinstitute.github.io/picard/), the methylation ratios were determined using a Python script (methratio.py) distributed with the BSMAP package. BSMAP is further explained in Xi, Y. et al., BMC Bioinform. 10, 232 (2009). For all further analysis, only CpGs covered by at least ten reads were considered, which resulted in 6706643 CpG sites.

### Differential Methylation Analysis

Differential methylation analysis was performed using MethylKit3 (version 1.12.0) between the halal and non-halal group. MethylKit uses logistic regression to calculate p-values and sliding linear model method4 to adjust the p-values to q-values. The use of the linear model method is further described here, Wang H-Q, et al., Bioinformatics. 2011; 27:225-231. CpG sites with an FDR below 0.05 and a methylation change larger than 25% between the groups were considered as significantly differentially methylated Positions (DMPs), resulting in 76339 CpG sites. 8000 of the exact positions of CpG sites are provided below in Table 1.

MethylKit generates a ‘prcomp’ object, which can be used to extract and plot the principal components. The use of MethylKit is disclosed in Akalin A, et al. Genome Biol. 2012; 13 (10): R87. Principle Component Analysis (PCA) is a dimensionality-reduction method which can transform large data sets to a few principal components. The first few principal components generally retain most of the variation present in the dataset and are useful for emphasizing the grouping structure in the data

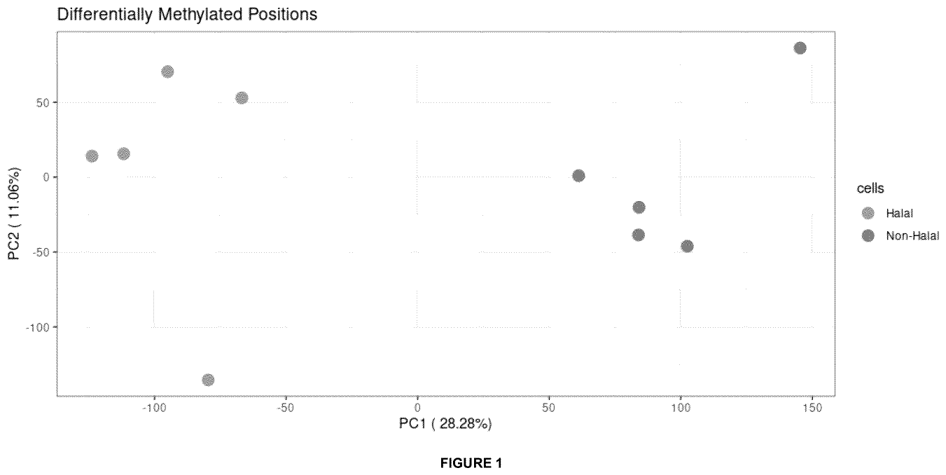

Principle Component Analysis (PCA) of CpG sites with min coverage 10 in all samples: 6706643 as shown in FIG. 1.

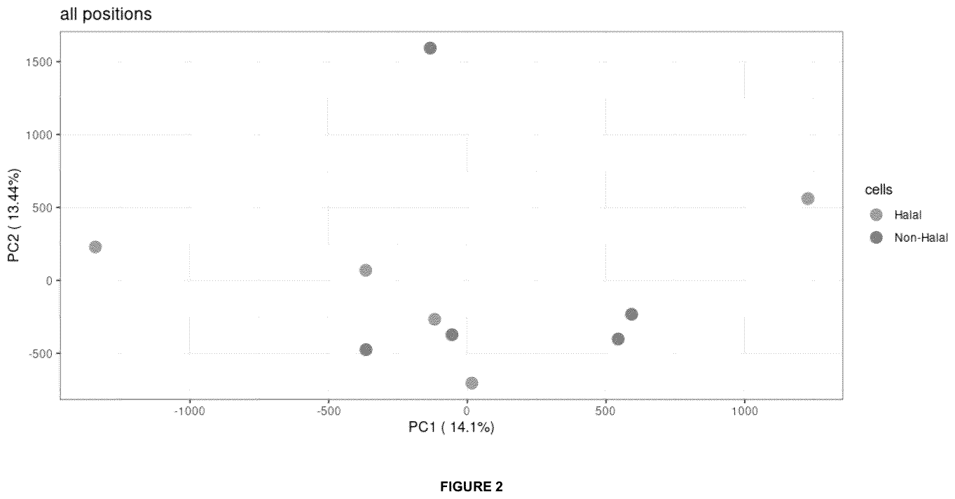

Principle Component Analysis (PCA) of 76339 DMPs identified is shown in FIG. 2.

Plotting of the first two principal components of the CpGs before and after differential methylation analysis reveal meaningful clustering of the samples. Differentially methylated Positions (DMPs) were able to effectively cluster of the halal and non-halal groups in a meaningful way.

TABLE1
8000 CpG sites of the 76339 significantly differentially
methylated Positions (DMPs) are provided.
Chrom. 1
91445266
91867071
91967201
91981222
92063881
92069641
92073135
92081175
92083695
92131882
92135148
92165423
92167401
92307934
92312695
92313276
92332108
92333906
92352272
92357970
92361744
92377300
92582465
92593964
93905272
93974796
96115407
96371496
96538914
96915466
97860771
97863639
97876042
97880230
97883788
97891514
97893212
97893439
97899631
98446275
98692606
98960666
99117578
99121881
99232390
99252123
99287791
99289823
99359371
99421194
99421858
99448844
99654329
99716835
99785545
100917096
100930333
101101457
101292685
102743846
102744036
102864597
102880419
102903166
102962907
104063989
104568595
104568620
104595133
104631656
104632876
104818162
104975981
104982811
105266058
105285746
105295755
105321097
105368474
105381938
105560437
105635166
105640239
105642157
105673237
105676895
105681427
105681484
105683863
105692217
105698330
105699206
105699576
105699772
105707513
105708851
105710899
105713109
105713111
105715985
105718836
105721776
105751431
105752308
105753944
105762703
105781437
105792078
105805436
105867020
106283398
106294434
106425948
106893845
106939542
107744685
108301785
108366607
108486147
108778228
108779637
108780962
108851200
108857042
108926099
109935317
110665181
110722557
110722814
110839940
110840818
110842089
110842917
110861722
110861914
110946514
111073219
112207254
112330478
112456912
112458680
112459051
112496515
112665311
112700276
113136471
113762134
114439059
114460607
114575194
114916845
115396424
115621595
116696917
116697823
117927019
118017977
118033433
118089911
118093848
118131172
118136901
118161147
119008607
119124231
119344316
119355404
119368819
120230511
120422623
120625111
121178764
121397345
121647893
121807900
121824142
121824210
122512052
123049523
123049532
123052329
123068223
123107969
123114294
123349475
123651857
123926705
123983999
124148639
124452290
124622057
124712952
124853158
124863910
124867306
124883595
124918230
124923315
124947741
124965095
124967278
124969058
124997352
125006439
125006751
125014340
125033684
125034173
125054934
125082473
125085464
125090877
125099564
125147921
125148474
125150325
125153852
125173073
125203179
125208804
125254337
125272378
125339873
125357321
125359392
125385361
125395754
125464756
125469495
125632584
125632618
125669657
125683261
125698733
125701101
125701110
125718359
125743607
125746036
125774134
125842010
125858515
125861404
125866546
125874319
125884727
125909227
126172521
126205734
127083857
127135775
127162145
127220720
127884768
128120980
128157121
128157475
128157479
128173885
128384436
128475164
128475292
128480110
128596220
128684206
128704202
128751939
128767268
128794428
128854579
129374860
129476510
129899761
130012254
130013586
130245492
130319008
130319030
130563065
130601476
130605109
130639300
130659430
130744891
130754343
130837123
130860788
130861309
130944180
130970381
130987860
131025990
131028222
131072082
131133094
131261309
131676536
132051650
132287014
132650315
132656796
132660602
132695582
132704388
132726536
132734920
132737490
132738992
132740165
132742025
132768899
133210571
133211140
133782071
135426887
136041303
136436514
136767040
136866704
137326517
137342012
137377490
137489393
137519343
137519629
137536995
137547145
137598436
138186463
139108004
139132147
139136235
139187491
140650814
141277439
141578386
144343642
144677569
144686073
144686075
144687798
144689304
144692492
144697582
144710006
144795578
144797224
144800504
144801281
144803670
145050426
145121981
145451463
145451945
145558268
145917456
145931042
145935377
145960083
146042234
146155876
146239148
146241420
146262117
146269043
146272958
146272963
146273025
146273165
146276681
146316583
146324512
146371548
146371885
146557657
149502730
150465132
150469057
150509218
150729602
152742456
152743469
153366893
153737073
154603274
154655198
154687086
154691612
154777790
155068874
155146854
155157964
155162576
155184262
155236895
155244554
155376402
155376442
155457057
155541042
155547013
155552669
155552719
155556193
155590671
155597149
155597153
155614129
155646644
155687883
155700657
155705203
155728974
155734370
155742615
155796365
155802167
155802662
155802700
155802743
155807953
155816817
155832450
155835629
155837307
155869890
156114990
156120715
156461197
156883113
156932232
156937170
156937205
157954117
160497936
160506368
162034557
162725506
162817783
163681567
163935687
165569236
165652675
165688904
165702788
165705257
165705497
165706063
165708998
165709890
165719595
165725246
165737545
165767103
165785447
165813096
165828727
165833022
165833212
165852402
165870330
165884647
165908252
165949160
165960057
165975749
165981078
165986182
165991867
166002795
166005089
166008648
166066883
166066885
166075584
166084033
166156845
166195280
166227531
166237937
166273533
166273555
166290052
166363283
166509975
166627303
166657038
166787396
166844749
166863787
166867092
166871664
166896225
167006063
167020474
167029214
167058495
167064270
167144246
167322775
167340574
167384596
167387257
167392147
167416805
167440362
167496487
167693272
167723808
167871832
168283934
168290126
168291845
168717194
168760859
168763622
168763727
168773306
168803179
168820464
168823558
168831438
168832762
168832951
168835755
168848172
168848287
168864935
168889014
168896771
169823660
170334839
170738323
171438730
172188483
172211646
172217260
172222643
172355058
172471079
172747295
172760272
172779423
172780043
172804974
172807045
172823336
172847449
172935802
173296397
173409509
173652186
173652205
173685545
173686327
173758323
173784152
174060181
174120860
174135482
174138971
174142132
174964928
174982194
174982982
174990371
174991305
174996647
175036662
175355317
175359960
175402204
175407107
175461006
175982267
176466520
176624543
176707023
176729772
176745759
176749986
176842055
176877884
176883812
176898804
176899097
177268589
177365734
177670328
177836006
177843900
177915886
177920895
178013012
178056608
178064177
178102305
178300407
178358636
178454173
178593890
178607269
178616238
178667962
178679298
178680112
178742965
178751309
178818120
178872338
178900345
178929481
319264
319410
325877
536093
536345
627301
760296
762340
816991
817207
819510
966735
967950
970186
1035896
1196465
1224151
1332268
1332298
1560766
1572692
2184973
2223029
2359937
2404907
2479336
2558671
14382900
14389729
14390869
14827051
14929225
14939706
14991334
15028631
15383339
15413268
15413486
15414018
15441150
15446382
15569788
15569827
15569836
15570253
15570408
15570654
15619089
15678236
15681449
15817117
15833532
16072651
16159153
16161819
13203436
13513341
13842665
16190183
16211498
16233722
16351085
16475807
16732543
17652483
18735623
18735836
18853357
18882209
19571377
19653777
19773907
19779796
20090531
20179912
20276645
20495256
20517274
20518370
20542698
20560247
20568240
20659139
20691502
20706689
20721742
20726124
20728221
20839659
22273004
22383399
22432909
22444851
22829183
22830906
23211382
23214235
23241308
23243334
23389156
23430635
23475513
23510371
23535151
23886388
24492604
24540042
24586546
24731438
24735099
24736481
24755549
24788889
24801771
24804092
24805622
24811417
24842468
24844813
24861179
24872169
24904631
25037418
25121168
25159242
25250046
25457060
25487976
25610648
25726621
25735114
25815449
26081266
26257838
26266839
26283110
26314674
26332721
26381297
28018540
28079758
28396011
28458656
28506098
28511754
29388678
29406214
29420752
29425871
29481199
29492316
29494062
29503894
29506513
29540786
29798029
29798122
30814262
30817126
30902502
31019561
31023575
31159741
31165440
31229185
31234922
31246294
31669614
31673649
31676662
32038248
32898097
32944698
33079318
33149190
33310670
33354426
33430313
33437899
33452207
33460714
33486676
33504940
33505543
33511405
33514781
33618883
33618913
33622227
33800890
33807512
33815171
33816062
33823134
33825963
33826944
33828409
33830242
33866193
33896238
34071524
34242912
34243235
34244681
34265922
34266766
34273980
34277298
34285700
34293155
34294268
34328685
34331314
34360401
34367358
34367986
34369759
34375737
34419897
34800629
34807434
34807494
34815288
34826340
34904861
34908454
34912415
34968929
34979301
35026718
35040247
35042167
35058812
35107913
35131529
35532743
35593161
35597126
35600404
35609623
35694665
35759719
35895326
36000827
36003303
36003821
36009155
36425979
36441177
36709235
36956526
37668404
37738780
38158166
38209414
38211833
38256200
38263027
38270069
38453831
38457853
38458213
38480797
38751027
38786161
40009588
40010022
40042184
40083721
40184719
40279892
40279898
40283834
40292039
40292077
40301100
40703875
40704872
40780899
40786977
40937655
40937724
41153687
41232042
42509687
42803267
42806884
42861799
43451329
44105812
44433795
44435328
44488974
44489348
44504564
44517830
44520699
44535599
44540873
45203012
45300374
45493366
45544163
45548565
45573355
45574076
45579246
45596364
45598141
45968671
45968738
46023343
46961140
47095504
47826288
47829834
47830834
47837438
47842930
47845133
47872740
47942262
47984939
47994249
48008071
48008408
48009224
48009595
48250341
48266831
49126782
49395228
49395373
49395380
49396739
49401382
49401636
49401729
49408458
49592638
49625443
49636050
49696315
49748603
49750804
49752685
49752727
49756470
49770452
49770584
49772813
49773055
50548561
50846057
50897467
51056433
51217041
51223225
51238060
51527783
51533958
51571819
51571821
51822619
53497262
53820967
54177600
54177729
54177825
54488266
54570344
54577178
54577204
54600294
54903804
56424858
56436427
56478275
56478320
56484791
56525590
56603282
56606169
56646011
56651642
56683984
56685242
56703433
56713290
56767962
56797854
56799167
56825555
56828515
56870539
57088541
57103690
57401969
57426080
57533254
57533563
57566537
57575258
57609045
57624847
57678644
57966682
58012380
58453223
58767768
58807510
58930361
59566840
59663274
59675786
60013635
60035065
60194921
60195054
60200064
60213764
60903793
60905155
60959821
61012939
61015549
61015958
61016067
61050127
61061390
61115472
61122984
61171043
61252627
61282558
61293140
61299141
61322710
61352704
61354683
61356272
61356617
61406500
61411565
61415067
61416595
61417597
61423258
61426885
61434581
61453250
61454235
61468355
61652607
61655714
61660240
61660682
61670981
61688220
61701762
61703457
61758580
61770481
61788357
61792162
61823899
61830342
61831903
61858546
61862361
63181469
63186159
63194132
63194675
63201679
63207102
63219318
63312846
63365888
63390824
63396791
63463582
63474034
63474968
63603762
63604475
63752360
63960451
64334285
64468916
64483339
64485126
64488884
64490304
64490311
64490359
64588633
64755245
65152161
65153019
65461909
65474561
65944762
65947664
65954298
65978358
65982810
65998400
66042103
66424490
67800000
68238392
68267055
69013526
69672098
70143358
70366189
70634997
71061807
71122924
71137161
72294095
73042226
73313437
76666897
76750006
76766539
77021906
77723553
78325592
78401374
78408343
78408659
78437879
78480970
78492171
78523151
78529281
78551518
78564632
78572048
78587234
78587971
78590983
78594727
78640074
78642873
78666723
78667269
78696868
78760285
78779583
78782640
78788572
78788737
78794889
78797248
78803274
78835840
78854386
78961153
78973493
78979074
78984418
78985071
79006380
79110837
79211477
79338850
79429849
79465679
79476613
79480563
79485098
79499733
79507776
80139879
80354462
80358495
80358807
80364856
80419655
80432561
80454289
80472311
80490453
80500621
80519649
80690588
80770170
80860477
81122493
81353754
81508354
81542318
81542814
81558833
81803936
83033841
83154255
83693380
83767136
83829008
83864982
83880380
84091498
84398043
84409963
84466434
84484280
84487152
84487792
84494215
84507048
84537342
84538419
84538500
84538607
84729282
84738520
85326600
86761516
86912968
87148298
87362387
87479163
87504783
87507863
87510726
87510885
88160371
88272698
88360762
88377020
88434126
88441401
88487729
88502211
88503144
88539744
88541912
88557969
88805521
88811890
88816794
88821807
88879884
88896993
88900804
88922149
88962164
88964954
88965668
88996531
88996607
89004067
89004244
89069332
91209811
91247958
91390020
91422638
91441050
178935184
179093706
179231596
180016240
180035845
180115098
180246333
180377111
180389226
180395259
180489883
180885351
180895054
180919468
180940180
182147089
182300991
182695122
183247434
183249091
183254344
183262775
183263456
183267094
183270436
183324367
183325914
183327645
183337763
183354204
183400500
183409964
184506915
184536318
184563246
184566349
184569970
184603205
184931310
184933514
184956416
184959146
184960702
185052255
185113253
185130118
185338469
185344911
185360778
185729471
185953948
186040093
186041129
186044977
186046856
186195777
186195818
186198168
186217862
186436398
186464164
186472273
186588446
187143754
187153184
187178826
187180547
187185160
187347036
187347074
187397637
187397738
187475404
187478343
187497762
187508004
187545954
187546161
187546939
187564624
187577852
187628995
187653228
187673682
187677452
188199645
188210592
188229817
188233031
188688218
188714714
188761112
188915443
188970953
188984548
188996607
189051202
189108134
189402169
189533755
190322146
194236651
194424939
194525283
194775374
195133015
195196652
195247099
195256837
195260880
195272185
195302794
195416687
195594509
195600380
195604257
195611553
195649873
195853868
196022342
Chrom. 2
617577
627994
686157
687102
894186
1054300
1085813
1103198
1859968
2676514
3139549
3921684
3924627
3924631
4113728
4317144
4431866
4807977
4807979
4840053
4850470
4915125
5211451
5317137
5320407
5610185
5677389
5713625
6421007
6450121
6460652
6532333
6625188
6634928
6676127
6861051
6971437
7297682
7775106
8241995
8431706
8466193
8503702
8537814
8629022
8637695
8639327
8650091
8678299
8711925
8717144
8736870
8736878
8745443
8747718
8810186
8834895
8940557
9120505
9423305
9423714
9508361
9557312
9576905
9899878
9926783
10012989
10086057
10104297
10239772
10246694
10254386
10264600
10289767
10294011
10294132
10311792
10312505
10345623
10358374
10372991
10395904
10396532
10396534
10404743
10447028
10890654
11305220
11408053
11787355
12207719
12262153
12293408
12294310
12339025
12377578
12456369
12467871
12605338
12728951
12729346
12757168
12827730
12851553
12852434
12866195
12887313
12887332
12895765
12897408
12903242
12911116
12918131
12918422
12918994
12919659
12920588
12925238
12972099
12993256
12995811
12997289
13000635
13026756
13030874
13041817
13164424
13171795
13177056
13177286
13178818
13375983
13380955
13450035
13468553
13471429
13473850
13894761
14103224
14118037
14118147
14198176
14565971
14841421
14900665
16143202
16154160
16160538
16164463
16208379
16257917
16260985
16266366
16306304
16306846
16316769
16393543
16411293
16493259
16502663
16502848
16503581
16512359
16515570
16577673
16581347
16603668
16624787
16628187
16682107
16731493
16757274
16858094
17083072
17366959
17469538
17509730
17539444
17540493
17558054
17558185
17558192
17641096
17661790
17798427
17803306
17928554
17928728
17928975
17929327
17930401
17934097
17947252
17969816
17972078
18563155
18563157
18582525
18711921
19182555
19182601
19185504
19186142
19186181
19186533
19194441
19196287
19293391
19514328
19560596
19645285
19685407
19697713
19712150
19712178
19731818
19791666
19842369
19846932
19969073
19969077
19979228
19981088
19982385
19982869
20013226
20264574
20264968
20341761
20348702
20350450
20351346
20362627
20378967
20386214
20388375
20389319
20390103
20453886
20531361
20537381
20538107
20539665
20587257
20608175
20655215
20661600
20676194
20678116
20681936
20685459
20690824
20693997
20697051
20698983
20699346
20707676
20707697
20714206
20722564
20726132
20756529
20757554
20775017
20778146
20799775
20814801
20818433
20823587
20824801
20826851
20873079
20894750
20913887
20975370
20982454
20983870
21010028
21012268
21029441
21101647
21120174
21203624
21205532
21208151
21215663
21217413
21264920
21291660
21296298
21297523
21300076
21300644
21480949
21490187
21490773
21503565
21557435
21583669
21588711
21633416
21662069
21737029
21808448
21830199
22280504
22293582
22515837
22518734
22520447
22616757
22707444
22991260
22998197
23051591
23086463
23139515
23201991
23277268
23277411
23398609
23566857
23675004
23682266
23793979
24020924
24021703
24036225
24128428
24259614
24269727
24289115
24328024
24345189
24345392
24444989
24952873
25012766
26161912
26185019
26665331
28174482
28186131
28198642
28214272
28277384
28374122
28415532
28432289
28593362
28632980
29099176
29321032
29375078
29605128
29710541
29759980
29816341
29830320
30238250
30245909
30259581
30509177
30567399
30610384
30611008
30623401
30624153
30633307
30639874
30660843
30676205
30694263
30873805
30883758
31025981
31061493
31069721
31070634
31198306
31198515
31205590
31210078
31240767
31247666
31248282
31257750
31261144
31261157
31261516
31271565
31291977
31293681
31301316
31302664
31304823
31306200
31323773
31323802
31329446
31333644
31334915
31335279
31344787
31354409
31354486
31366875
31383418
31386887
31390626
31390826
31395814
31399609
31399673
31434648
31442768
31444085
31480087
31484937
31512101
31515003
31515964
31530506
31571483
31577762
31578391
31579637
31594402
31616473
31621223
31625459
31655443
31657530
31659601
31660352
31660552
31660566
31684252
31684272
31704536
31749204
31992670
32027940
32034651
32036641
32067182
32072243
32081133
33438049
33447193
33876382
34269185
34939482
35632134
36210198
36393978
36410630
36428206
37070305
37095159
37102054
37108186
37113095
37114790
37121126
37121956
37122035
37122071
37129870
37133346
37189598
37191404
37194032
37233991
37375383
37377419
37385757
37386669
37406416
37409926
37466035
37491435
37491995
37494587
37499667
37507582
37521340
37530622
37531253
37548939
37635921
37645505
37684662
37691829
37696575
37707312
37707390
37778869
37796553
37801260
37808969
37936016
37992447
38028413
38078824
38368866
38396521
38563601
40455662
40458865
40489845
40494766
40494816
40625163
40722157
40788783
40850273
40883132
40929050
40977901
41284791
41336628
41418647
41617754
41643329
41717901
41910968
41912840
42154885
42190574
42356641
42412792
42676149
42735703
42855942
42875014
42884575
42991101
42991201
42992138
42993029
43130467
43608952
43722767
44056670
44057247
44057319
44127579
44150346
44151147
44185413
44299639
44620202
44674593
44712401
44718031
44736898
44737993
44752230
44752709
44762441
44816361
44816408
44829102
44831568
44837780
44838540
44841449
44841598
44858353
44860484
44871207
44886415
44894321
44894539
44904631
44912959
44921810
44923168
44940610
45090140
45194429
45271427
45292472
45321378
45330866
45505777
46107511
46878458
47071322
47642423
48602780
49101356
49110506
49125051
49170761
49186559
49214895
49216357
49225161
49232405
49237702
49249130
49258494
49278842
49489963
49915973
49982864
50352779
50437667
52044431
52242760
53419227
54093853
54157615
54333381
54399445
54406773
54408434
54415298
54417195
54429108
54429178
54431881
54434683
54435698
54435717
54446105
54448418
54545367
54635369
55325000
55328372
55366660
55652487
55652499
55731429
55755398
55793079
55806793
55927240
56106914
56161169
56222275
56232074
56236322
56245031
56245047
56246665
56252341
56294082
56294978
56339730
56479782
56699632
57446998
57466411
57598780
58036238
58050619
58058093
58437345
58447213
58483160
58492932
58493805
58496721
58501872
58986726
59818083
59993715
60331426
60683508
60924721
60968298
61024275
61027734
61033140
61057058
61083232
61084800
61118070
61120977
61146381
61487474
61501061
61501326
61505153
61712033
61775073
61864801
62132390
62132683
62252271
62259321
62357625
63319502
63359221
63988409
64161771
64360539
64362396
64363502
64394714
64669366
64937881
65511258
65949551
66495915
66497871
66547060
66553647
66560781
66564350
66934554
67430872
67480178
67544093
67649862
67851665
67860596
67861328
67872632
67873708
67878733
67878874
67897660
67912058
68088862
68253510
68364197
68377568
68444526
68466195
68536666
68536830
68609158
68631419
68636448
68639436
68647171
75710990
75756384
75835302
76828911
76989531
77451846
77470725
77481746
77562252
77563146
77810343
77854573
78336294
78537976
79360904
80361385
80490518
80504281
80589672
80697035
80718940
80768356
80768746
80769015
80785933
80786143
80786625
80799784
80800970
80811879
81112887
81112893
81171184
81251181
81590118
81643771
81679675
81803447
84264541
84273546
84306959
84353540
84487380
84490034
84516953
84552901
84705766
84773623
84786085
84797052
84812949
84814308
84925891
85095899
85222845
85431076
85431278
85875135
85875150
85961546
86127335
87718293
87722490
88211110
88227285
88286617
88291960
88789027
89343567
89361065
89491513
89522439
89818616
89821854
90028892
90151228
90515684
90515689
90554149
90557417
90568641
90569921
90570190
90584505
90587390
90613232
90626145
90637707
90649957
90654245
90659553
90663516
90669342
90675052
90676352
90700211
90732758
90744492
90825932
91312967
91605497
91630213
91721543
92066863
92473479
92488264
92503177
92528655
92529297
92534929
92540893
92558791
92691787
93121694
93136437
93182607
93188478
94003621
94015336
94043096
97313276
97322739
97334712
97504238
98814601
98937199
99100879
99563155
100439721
100605760
100606750
100622799
100680360
100690766
100789599
100812285
100821829
100841226
100947288
101220501
101267709
101290883
101296328
101307298
101310238
101312818
101434936
101435962
101535193
101545949
101646992
101762867
101801291
102103854
102107126
102112140
102252232
102252241
102253223
102253453
102323102
102402108
102559425
102603682
102709526
102709970
102753243
103057090
103091998
103446498
103448110
103461050
103466928
103473904
103498548
103555524
104061256
104110404
104283527
104293293
104347875
104364531
104391673
104395447
104426917
104469826
104596379
104605202
105051158
105067124
105068868
105094733
105138635
105287251
105298814
105301845
105307742
105309588
105311110
105312387
105313210
105346291
105367288
105382540
105407646
105408406
105506658
105513813
105535650
106304350
106310238
106446886
106703235
106769835
106893610
107070707
107551907
107575679
107713730
107716147
107716185
107853691
107886809
107888449
107888454
107888461
107891906
107891937
107952258
107958967
108330355
108330357
108342913
108379315
108406157
108413888
108428474
108485286
108494137
108503729
108504558
108506985
108516762
108553313
108567288
108587965
108602718
108631704
108633770
108634765
108682464
108707779
108756831
108783858
108795154
108807754
108848029
109709060
109713123
109713620
109717330
109718019
109757144
109802971
109816180
109877589
109877760
110083973
110152368
110206311
110690434
111204781
111204865
111204887
111205134
111215039
111229134
111231608
111426183
111573956
111663874
111884287
111964851
112052101
112052479
112109258
112125484
112163563
112165319
112221165
112235818
112236610
112237276
112237348
112237765
112267558
112371071
113099371
113099457
113137986
113142568
113168453
113235153
113253375
113279712
113279804
113296967
113318632
113329616
113455493
113490915
114003127
114018685
114041533
114195149
114494457
114809683
114827295
114833054
114834810
114882898
114941647
114955133
115002162
115248380
115272668
115273931
115312835
115358049
115358053
115465478
115549279
115627235
115699223
115932462
115932899
115934046
115938577
115940351
116076068
116112531
116136591
116147793
116539507
116649953
116649961
116703193
116719424
116842818
116884897
116928607
117120080
117156869
117215654
117305969
117307745
117307893
117308293
117321527
117321559
117370048
117445537
117817923
117823077
117925722
118005622
118006362
118009072
118013210
118020735
118021205
118110010
118168314
118185368
118242597
118243183
118269053
118370154
118370249
118375023
118378417
118419413
118425112
118482940
118485846
118487207
118607547
118618309
118641482
118685718
118785822
118843297
119366314
119970452
120654043
120842851
121289713
121828201
121954277
122000121
122199967
122331606
122427173
122436224
122475489
122767178
123043254
123140548
123167826
123168950
123174245
123206194
123247711
123372302
123384754
123386121
123405271
123406913
123409299
123414059
123419730
123450378
123480459
123484604
123541197
123547486
123567187
123578628
123592723
123593917
123613330
123616025
123616262
123616283
123617803
123620300
123623354
123628384
123639459
123647992
123653469
123744217
123778177
123956852
123996457
124124902
124176964
124203619
124212323
124212348
124260912
124261716
124262248
124266967
124317801
124319785
124322133
124343784
124728107
124861307
125025517
125025571
125035276
125064396
125075063
125103088
125166326
125187885
125188539
125204406
125219161
125222852
125226031
125234961
125235414
125295538
125500717
125785756
126244057
126318248
126323195
126467806
127229328
127360312
127384429
127384614
127415259
127561234
127574512
127578300
127591004
127604230
127604367
127605802
127684426
127734396
127736644
127766766
127901602
128114098
128114408
128142897
128163450
128166268
128259528
128340431
128390156
128410603
128441224
128448310
128450308
128451065
128464001
128470324
129709730
129727160
129730866
129752086
129753280
129762505
129770939
129785285
129900636
129928156
129928795
129929046
129940238
129969424
130008209
130016323
130057220
130067112
130368839
130371104
130421881
130441680
130512916
130710959
130717908
130725363
130849576
131008009
131116599
131155170
131607305
132411096
132439144
132439564
132461126
132512945
132524222
132527325
132545356
132669593
135150654
135881725
135996684
136283388
136335147
136347284
136407016
136558035
136577908
136808953
137809614
137959277
137974778
137980544
139545670
139554061
139554785
139577628
139581419
139672963
139673759
139675304
139678760
139831992
139835206
139965495
140291974
140351366
140500910
141390548
141762744
142655633
145004166
146391216
146451132
147078436
148773696
148784530
148963613
149026048
Chrom. 3
1341734
2108765
2140740
2350262
2437016
2552871
2576549
2592899
2599433
2602855
2631414
2741237
2761118
2814685
2840097
2840357
2840575
2842261
2847740
2859288
2868686
2914188
3013397
3675677
3932222
3932426
4218622
4291130
4295351
4298499
4319495
4471021
4473095
4494086
4495676
4498864
4521830
4551590
4666877
4680535
4685429
4686117
4736841
4790405
4801727
4812900
4813177
4816797
4821646
4826038
4828684
4831925
4835925
4847593
4856879
4859198
4887981
4911035
4949123
4949252
5007904
5083660
5090362
5127658
5436749
5489355
5579065
5607123
5610422
5610563
5618656
5916254
5989867
6016462
6349033
6671380
6938099
6980855
7139839
7261188
7504658
7770923
7776788
7776841
8329155
8404188
8546281
8794813
8871725
8957784
9016764
9031456
9037919
9044675
9051098
9071424
9194213
9322309
9686221
9834030
9862360
10215937
10974238
11035812
11081330
12538788
12544640
12557063
12559197
12566726
12567432
12568879
12569410
12571758
12584194
12609626
12631052
12641176
12643368
12747548
12784226
12848801
12921481
12995012
12998472
13003762
13021427
13133915
13134241
13144679
13151775
13156671
13185398
13359985
13731025
13846784
13861957
13973485
13980409
13981397
13999554
14003137
14008502
14024092
14026483
14034875
14036571
14063651
14074694
14099890
14122142
14122758
14123465
14123807
14128230
14132450
14136398
14136618
14140261
14145718
14147270
14153203
14247221
14573109
14751214
16129875
18077999
18737151
18879854
19210868
19250593
19327532
19329480
19333227
19336300
19353449
19414773
19638991
19748286
19748689
19775082
19790194
19817241
19864785
19866636
19872698
19874591
19874609
19899128
21682286
22090509
22104411
22107785
22109459
22126745
22139064
22141239
22198937
22202587
22204214
22264915
22350095
22350423
23014067
23056829
23072493
23073767
23073839
23078834
23080660
23110596
23111672
23112050
23154938
23179695
23190662
23226500
23253315
23255610
23258908
23258990
23268865
23295578
23300112
23300983
23368271
23368287
23470530
23510180
23512869
23516714
23553482
23554144
23558980
23559716
23562345
23562451
23604920
23613193
23613312
23662734
23664082
23698063
23718848
23738914
23755767
23758954
23771023
23774332
23813964
23817499
24639761
24760193
24897479
25565667
25794072
25819295
25870009
25875450
26065434
26070473
26077200
26196666
26225862
26234140
26246746
26246778
26280181
26281537
26341162
26530336
26532134
27238694
27245442
27251679
27253129
27264409
27285104
27360484
27362714
27364753
27371496
27519370
27733502
28861778
28861821
29841072
30120040
30149659
30275103
30278423
30444739
30600691
30772965
30774605
30774746
30775255
30800939
30819906
30824033
30833969
30923624
30933321
30938391
30981441
31257798
31263619
31396208
31402457
31407454
31410048
31413002
31416605
31445240
31463238
31468258
31469489
31469693
31487275
31505392
31532349
31543906
31596358
31603883
31696328
31700844
31719543
31734371
31737914
31752292
31837002
31841880
31842337
31910441
31935171
31935210
31967745
31984406
31993870
31993899
32332823
32400119
32445455
32446815
32451989
32517246
32781671
32781688
32784033
32792993
32876504
32919424
32919879
33370018
33570828
33688914
33946497
33963104
34171220
34325038
34345873
34500042
34763463
34773492
34827482
34827973
35263825
35393598
35393822
35406957
35409552
35411090
35439181
35483594
35491858
35531727
35559333
35622685
35642085
35738532
35745982
35746714
35750637
35750643
35777924
35830122
35836525
35847560
35856075
35861008
35888051
35926075
35926247
35934245
35938019
35973435
37898064
37898085
37940877
37955813
37956094
37962655
37989297
38000127
38062649
38181323
38185812
38311346
38312173
38317620
38317626
38806425
38871552
39032588
39062227
39120436
39123904
39124988
39125288
39125293
39125373
39129774
39145829
39251239
39266909
39358099
39363536
39366682
39366932
39372059
39395494
39538776
39579790
39588564
39589004
39589644
39595693
39603828
39746916
39785500
39854322
40066274
40138367
40536071
40713583
40713783
40963028
41147125
41263809
41386294
41386623
41961084
41973430
41973693
42235050
42273865
42302757
42359049
42374915
42387751
42400633
42403419
42431181
42434808
42453844
42463803
42543199
42614792
42775059
42801413
42870587
43714584
43904223
44095760
44174316
44262946
44431230
44431402
44436814
44440606
44462331
44492364
44509842
44513512
44533443
44593550
44715504
44857107
45265574
45689952
45910332
45993418
46040623
46079833
46125714
46142392
46144761
46163430
46167772
46167896
46171403
46222747
46227559
46234519
46234822
46236793
46238364
46246558
46253767
46271487
46271803
46272168
46275373
46282773
46283327
46316722
46320753
46374748
46474198
46623611
46625653
46629602
46648344
46650435
46651471
46652784
46653098
46659147
46659663
46775137
46786892
46998243
47005596
47031522
47037712
47113899
47117427
47149250
47167155
47183287
47183292
47183349
47281646
47296453
47296470
47494348
47552816
47564908
47621553
47637234
47645989
47650212
47650241
47700644
47720400
47726225
47726253
47742925
47748730
48024974
48091474
48128388
48144967
48145041
48147715
48223211
48241524
48300917
48413696
48414024
48415027
48429790
48440161
48467009
48503780
48505847
48505894
48526212
48533399
48533770
48536806
48538694
48538915
48540460
48545227
48545499
48666049
48671110
48689731
48715607
48733641
48791515
48804815
48863293
48875415
48880794
48896955
48902320
48904819
48908969
48918254
48962421
48999100
49056953
49064708
49066605
49074474
49130627
49135421
49153313
49155021
49157234
49405901
49482270
49509346
49522529
49555175
49557310
49560056
49674318
49678441
49680490
49683915
49706832
49707705
49714856
49739895
49753086
49772985
49804754
49844152
49844397
49956795
49985243
50031242
50204018
50210100
50213782
50225508
50225675
50227189
50227592
50240288
50247417
50255584
50294399
50295897
50299729
50299741
50299907
50302267
50309413
50315646
50332379
50337217
50522769
50535729
50896737
50898652
50934847
50964376
50968595
51110812
51131824
51212989
51250926
51290287
51293942
51320744
51330281
51373053
51374254
51374262
51374636
51377916
51378748
51380869
51382794
51384013
51424234
51654360
51660436
51695390
51853753
51871009
51873303
51873399
51926805
51941992
51970747
52026700
52132775
52132924
52144748
52260459
52322463
52359502
52368026
52369468
52473023
52631134
53210175
53326004
54079262
54103130
54474102
54652816
54653150
54664818
54752135
56447860
56499624
56847699
57779221
58114792
58219620
58289703
58664870
59540657
59580738
59971486
59982171
59997046
59997074
60010334
60100884
60403803
60403837
60414524
60531280
60681719
61983144
62028265
62381918
62470318
62474249
63122958
63144389
63144534
63167009
63474026
63489005
64002297
64012953
64039208
64042406
64136304
64866254
64959612
65071166
65086849
65214500
65225827
65235154
65250537
65252478
65262099
65263222
65562730
65596583
65612180
65717362
65739568
65740426
66200672
66273878
66337715
66538061
66563594
66573319
66595736
66616042
66616059
66622728
66623897
66662790
66662926
67099366
67212081
67353633
67555050
68023198
68026360
68146444
68163689
68600305
69146190
69154008
69179618
69339972
70960510
71002231
71013572
71064537
71630595
71743210
71943864
72739846
72788974
72794536
72811487
72999617
73077899
75485813
75586132
75853782
75865428
75898447
75925045
75973521
75976435
75978183
75992508
75993565
76003384
76055456
76068313
76068441
76068577
76100851
76114999
76123968
76125562
76134475
76138127
76139943
76145372
76150654
76151732
76152163
76174484
76175313
76175886
76185043
76226716
76228455
76229899
76231105
76239254
76246851
76250239
76281378
76442812
76442819
76469801
76470108
76535272
76741382
76755036
76777689
76844490
76918027
76920662
76988512
77063993
77201226
77202363
77211912
77214382
77276237
77393379
77439773
77537564
77589780
77712151
77714717
77734175
77882891
78025879
78044047
78126954
78210325
78210335
78289508
78336825
78341224
78343739
78350555
78369323
78553716
78556624
78560251
78605469
78697465
78724131
79196813
79392673
79392733
79453485
79558632
80146819
80653964
80666908
80824512
81196559
81309274
81383168
81396470
81398556
81412826
81417156
81421513
81429019
81437376
81448101
81450572
81462851
81472255
81531341
81534736
81925541
81927909
81935335
81942664
82043898
82067803
82080089
82094768
82123431
82124649
82127389
82128329
82129512
82129860
82131419
82140739
82144480
82885415
82926903
83087378
83164543
83431165
84081880
85079029
85443087
85476953
85501629
85882521
85976388
87011733
87034543
87114028
87302037
87302631
87307053
87333604
87335943
87381235
87382507
87409662
87411263
87419300
87434841
87439675
87453505
87467710
87472976
87477139
87485554
87506555
87527771
87533334
87640387
87803316
87810675
87812435
87812608
88511371
88512731
88523225
88530183
88652155
88695523
88705007
88783780
89470619
91561988
91635780
91640415
91640456
91642542
91645080
91659037
91664512
91681603
91877547
92066303
92155819
92165306
92175105
92317171
94464442
94503288
94665019
94838389
94935971
95446881
95477675
95485321
95485332
95502514
95632099
95745297
95785288
95788276
95797254
95801405
95801885
95839336
95867464
95928991
95949228
96015348
96019922
96209231
96357787
96520587
96541523
96752978
96785033
96785059
96785313
96785759
96807314
96815673
96849613
96867087
96891219
96983950
96994883
97027498
97047634
97227200
97342164
97344850
97410888
97422646
97645775
97648912
98082728
98230766
98509066
98585150
99520472
99962623
100647057
100651714
100893826
100918033
100919720
100943944
100949300
100951959
100952158
100964417
100966517
100966563
100971797
100975971
100976572
100977372
100977902
100991622
101001735
101100708
101138285
101143537
101820219
101859859
101871167
101894075
101914786
101930354
101933834
101935194
101935577
101937510
101943963
101954081
101961082
101961948
101976103
101978654
101979489
101984966
101993816
102006897
102017198
102022028
102033544
102036031
102063288
102110056
102111316
102116877
102122555
102180988
102225590
102252400
102304688
102331191
102335028
102342541
102416478
102457501
102670290
102683948
102788672
102800961
102839667
102881835
103487881
103489557
103535748
103561942
103577669
103622366
103632714
103721551
103728982
103826990
103872420
103901166
103901571
103936536
103939404
103940333
103957928
103962024
103964239
103965424
104028433
104073406
104106589
104177705
104177857
104180058
104218456
104219876
104230558
104230841
104233246
104354459
104360068
104403523
104478383
104520137
104682686
104687596
104697003
104709139
104725604
104725868
104730337
104803944
104846074
105094882
105109642
105264607
105269694
105276062
105280354
105293278
105407197
106198847
106597000
106615492
106615656
106685208
106814027
106830276
106871262
106911376
107022680
107035471
107225607
107258101
107290471
107401466
107456864
107482932
107496388
107815570
108274105
108417594
108535460
108551494
108855342
109028565
109049665
109097026
109340894
109435484
109465253
109504849
109624644
109637985
110131140
110139549
110301788
110308277
110369260
110408827
110505635
110511297
110513383
110513907
110520161
110520554
110521359
110524714
110525651
110528977
110530918
110536340
110544274
110554527
110728409
110739847
110751757
110752251
110756132
110794610
110856505
110879647
110890201
110910883
110977496
110978143
110994610
Chrom. 4
2393346
2423390
2522658
2745027
2832015
3035653
3247811
3256747
3532834
3670174
3676677
3691313
3934080
4223288
4259069
4273542
4376577
4549956
4555528
4900235
5005124
5014402
5033626
5099377
5258684
5291953
5990521
6143114
6208926
6308397
6369159
6668564
6686115
6903881
7605278
7740658
7749672
7757881
8372477
8451269
8475947
8536038
8536081
8536418
8688665
8698637
8818574
8857391
8891868
8922774
9673918
9677661
9679254
9684437
9709468
9715041
9733668
10640067
10802755
11052632
11118022
11124334
11128413
11281878
11303002
11303061
11317717
11512921
11548100
11583438
11587985
11601835
11618567
11644034
11659671
11672201
11680543
11685923
11721144
11740649
11769886
11890919
11906188
12384223
13225569
13272250
13388666
13393404
13549445
13603452
13611330
13701023
13968390
13970172
13972124
13986888
13989156
14005697
14012346
14022416
14040971
14080695
14185108
14199039
14233529
14256980
14410151
14517991
14526832
14557453
14589723
15024892
15907350
15908685
15933730
15939372
15942119
15942668
15959360
15989244
15994342
15997543
16000642
16009548
16009676
16015412
16464095
16465102
16780164
16780187
16874515
16888157
16934486
16936429
16981136
17013840
17029106
17041731
17515105
17623485
18280648
18526756
19508173
19627949
19808842
21049305
21145170
21328510
21359971
21632424
21855938
22432047
22604988
23444353
23466652
23515532
23564663
23566676
23571474
23584797
23598276
23607798
23609780
23629000
24075469
24354394
24954245
25142257
25142285
26316335
28272091
28465687
28803221
28900528
28902710
28903046
29169207
29374430
29546377
29548870
29550966
29826293
29895285
29895761
29941181
29986243
29986252
30222436
30247108
30458520
30574804
30587542
30605751
30663108
30823131
30915108
30984252
31074512
31295742
31304140
31315426
31378277
31381190
31474361
31474731
31476264
31497953
31537185
31537219
31634271
31730761
31981541
32206623
32259121
32279372
32280162
32379851
32412221
32430540
32451858
32454316
32514044
32566081
32642913
33043370
33134909
33350483
33350502
33471512
33501320
33586679
33664988
33665560
33673334
33677782
33692426
33692511
33736812
33815129
33855921
33869511
33966460
34038368
34050041
34093161
34170739
34263581
34340098
34352681
34371688
34391696
34558799
34655224
34778588
35122795
35348553
35353955
35359534
35364131
35434002
35435113
35441486
35453773
35482092
35485753
35493895
35724188
35783348
36694686
36837536
37338983
37409932
37437688
37567006
37690431
37695440
37756808
37757657
37763118
37763519
37769267
37775440
37779154
37785557
37883321
37893085
37980709
38160430
38170264
39031602
39330032
39435387
39461339
39461374
39497697
39537311
39538854
39545361
39581944
39772367
42535418
42540528
42585311
42958992
43100016
43139811
43375329
43420951
43472795
43483536
43488234
43840612
44572189
45026404
45244572
45306142
45335143
45335584
45361936
45375764
45459769
45547834
45558221
45570182
45591966
45593108
45600980
45603291
45627119
45803251
46037821
46442709
46541773
46544506
46761541
47195615
47235520
47242781
47245303
47248073
47257210
47310842
47338685
47344491
47556709
47578595
48674011
48797284
49048767
49049458
49087268
49155886
49195434
49512441
49917041
49921191
49930019
49933414
50054460
50093373
50095737
50101108
50179096
50195827
50198199
50207820
50208459
50209176
50219198
50223743
50265756
50436592
50445678
50450590
50451326
50464491
50464786
51048234
51454889
51493247
51493523
51615210
51682335
51721985
51727942
51851197
51975051
52148153
52564675
52879369
53840125
54372202
56842774
56893199
57475733
57499097
57513085
57532309
57537194
57539608
57572893
57587739
57631457
57653927
57735514
57785090
57824603
57839286
57895254
57900689
57906285
57924600
57951189
58067950
58079006
58116635
58135808
58140362
58149745
58149868
58152324
58169737
58181047
58209501
58288346
58324474
58659223
58660166
58660174
59682163
59994738
60165956
60178717
60283441
60612565
60873573
61089988
61348923
61356557
61390384
61648515
61738110
61755914
64116827
64134356
64462397
65118743
65226291
65239129
65277399
65295135
65336270
65337694
65379447
65383585
65898021
65900849
65973723
66103635
66108914
66135518
66136095
66169712
66178630
66178679
66606647
66618926
66822262
66826474
66830683
66831436
66864867
68986584
69059422
69062434
69451696
69520146
69542552
69601091
69601488
69628609
69796031
70964350
70977750
71025045
71111830
71145716
71202153
71227623
71259002
71304664
72099608
72109789
72111159
72272523
72293374
73712803
73912365
73993051
74004702
74085025
74144565
74456901
74564477
74606351
74623978
74643194
74732457
74879192
74913369
74972567
75465889
76489494
76586163
76775865
76779145
76841295
77063117
77088079
77117795
77478892
77487793
77515828
78029219
78042510
78800917
79116234
79435219
79654729
79664216
79967339
79984842
80037832
80062776
80066809
80066827
80068833
80308311
80442473
80531618
80826366
80979170
81018607
81019896
81019965
81079003
81205819
81332038
82433296
82439444
82440779
82571006
82577726
82684064
82742988
82752429
82864288
82874440
82875986
82884846
82916054
82917040
82958196
83025340
83159037
83407054
83748275
83903272
83917221
83918606
84151860
84227956
84461432
84658015
84696412
84830957
85080696
86861024
86907465
87116444
87276965
87615879
87767096
88107068
88293439
88464040
88474672
88474914
88593991
88611876
88611930
88632280
88664766
88920017
88921697
88970007
89053890
89257630
89259160
89314508
89434203
89671513
89869832
89886884
89978349
90040761
90268291
90268297
90269856
90269930
90484968
90492058
90492309
90493986
90495443
90495674
90504092
90505115
90515283
90518257
90527738
90529382
90545888
90547332
90549129
90591308
90630157
90637987
90666101
90781250
90795596
90804097
90829358
90887919
90905868
90939993
90961385
90961535
90974052
91008033
91073816
91225988
91262409
91262419
91262451
91262468
Chrom. 5
285436
480120
518760
898563
5180158
5357551
5483539
5483544
5487193
5627197
5779213
6444481
6749262
6945781
6945870
6945883
6945895
6946873
6947137
6947229
7112725
7138277
7153504
7158705
7567642
7659486
7677757
7681037
7697222
7733587
8353231
8388121
8440395
8456012
8465822
8474070
8479753
8506445
8515082
8522676
8541488
8558256
8883821
8894211
8900261
9042053
9053338
9076489
9170486
9288314
9453273
9456798
9535964
9536350
9541301
9701802
9771111
9788490
9956783
10162600
10254063
10275335
10500357
10500360
10500509
10500592
10500805
10632763
11116730
11322882
11772994
11777595
11799079
11830284
11843864
11865559
11967107
11970884
11975991
11980679
12118687
12118738
12256162
12272692
12638867
12843162
12953450
13258951
13429535
13937889
14006943
14310749
14332206
14374253
14374454
14479872
14793107
14795262
15227038
15229221
16103464
16165757
16222698
16248606
16465735
16465790
16544169
16580069
16580225
16650554
16754913
17097877
17411598
17476738
17504024
17780197
18521576
18524379
18619795
18669106
19085004
19158441
19526606
20041151
21134200
21387741
21548355
21663520
21667938
21669609
21846771
21879894
21902504
21902506
21928894
22778065
22808311
22915498
23037284
23109868
23268689
23387674
23416653
23429027
23474220
23478608
23482751
23485723
23536377
23562477
23562840
23694178
23695754
23720561
24095015
24159303
24178840
24230869
24301623
24355314
24826873
24917732
26229432
26231468
26277364
26300125
26325348
26358878
26375997
26395487
26628185
26804070
26843650
26975158
27025429
27819625
27867426
27876741
27892541
27904923
27905351
27912320
27917492
27921306
27925513
27925604
27925707
27926961
27946028
27946426
27957050
27974501
27996935
27997150
28045641
28088349
28092541
28093056
28144512
28169124
28178443
28182222
28208767
28216431
28218371
28252302
28253261
28266096
28288965
28294781
28320105
28346609
28360917
28367000
28368368
28375302
28387689
28399004
28439360
28460075
28479398
28494426
28495713
28495724
28495728
28499049
28522635
28549789
28579135
28635587
28650948
28671428
28677310
28677454
28745536
28772716
28780393
28790495
28792445
28794867
28873383
28957069
28976003
28987468
28993243
29285156
29840885
30422546
32212342
33318634
33365851
33995440
34103848
34751947
34785911
34841836
36186926
36188440
36210952
36407744
36444521
36452185
36453079
36460679
36476657
36530788
36535365
36535381
36599713
36729392
37244406
37393340
37400223
37416130
37589766
37685027
37718335
37736252
37811457
37859833
37893437
37897653
37992546
38312772
38609675
38623828
38643432
38981668
38983841
39017503
39170269
39348765
41025405
41054480
41088163
41366528
42217543
43490716
43491333
43509515
43556601
43593547
43602267
43605338
43863077
44072718
44074310
44242412
44393143
44561707
44602581
44614357
44618456
44635285
44635497
44636404
44638013
44640104
44640297
44653727
44720720
44770565
44771694
44785475
44844461
44992539
45021368
45027850
45028031
45037404
45038291
45039509
45270723
45288647
45785705
46220615
46295095
46372121
46633247
46680364
47171298
47513516
47844939
47960736
47968642
47970867
47986619
47995708
47995915
47995950
48005234
48005844
48017786
48025292
48026737
48046067
48046823
48051344
48051700
48051800
48059618
48064266
48075484
48079774
48080186
48091901
48106809
48111261
48112726
48144765
48144914
48148903
48148975
48238816
48516444
48735091
48738523
48746129
48746379
48757631
48803407
48842159
48946583
48952916
48960561
48996388
49014833
49153560
49153865
49172851
49184257
49184649
49199291
49216500
49454351
50839124
50878505
50893477
50903999
51047931
51048996
51216073
51721325
51791933
51840403
51854301
52175427
52649625
52654526
52659548
52663733
52686269
52687414
52739657
52746737
52767738
52769858
52812262
52818991
52820946
52826960
52843844
52843862
52846856
52851996
52941718
53024807
53031528
53114393
53127784
53136411
53136460
53164992
53169152
53169162
53175644
53198315
53227075
53245954
53248048
53248838
53250287
53253278
53253659
53254508
53259108
53263774
53263787
53264782
53264824
53297330
53299189
53307827
53308188
53309222
53311801
53316042
53316205
53347413
53350298
53363620
53365511
53366711
53367838
53370753
53370882
53372280
53373358
53373821
53373881
53373958
53376325
53376633
53377206
53379760
53381231
53390948
53399924
53412026
53420075
53420319
53429321
53430360
53433458
53433823
53441645
53442108
53446675
53448879
53454674
53461819
53461822
53463537
53466724
53467872
53469344
53470944
53476348
53496346
53499802
53500408
53504404
53504847
53505057
53566111
53568275
53967309
54573351
54591129
54594211
54595828
54598721
54781709
54861907
54893529
55116043
55126031
55127763
55148658
55150342
55183258
55612488
55752915
55798281
56074892
56284306
56386561
56456436
57274219
57608365
57764789
58003624
58040379
58238117
58257556
58263133
58344094
58563317
58563592
58595400
58606679
58632869
58688607
58691364
58704760
58707791
58725598
58744255
58890151
58890658
58891974
58956549
58971642
59352204
59360896
Chrom. 6
692734
1066199
1066321
1295055
1317745
1549720
1688316
1751703
1766425
1799861
1829961
1831413
1832444
1833736
1838966
1839531
1842351
1843581
1843645
2282080
2496618
2535628
2573835
2581214
2581713
2582533
2618198
2624866
2644275
2768921
3063502
3187511
3396271
3396276
3396299
3419176
3439338
3462007
3662611
3683701
4451463
4454145
4687447
4692777
4762220
4870197
4876925
4975292
5003289
5013941
5035438
5037117
5061613
5091778
5227438
5242145
5324181
5381946
5481759
5483099
5603835
5710720
5736217
5737206
5796834
5909340
5940725
5983112
6174972
6204887
6243492
6246540
6251296
6402282
6429832
6558093
6669093
6698506
6769427
7450801
7826925
7896752
7900195
8107243
8355283
8359024
29142506
29170831
29183045
29203352
29621056
30723388
30915629
30963710
31052460
31069085
31211983
31302563
31326918
31350235
31379349
31379735
31470098
31919184
32222841
32421797
32533531
32533539
32553836
33316952
33337923
33342133
33651448
33668577
33721908
33782061
33807947
33864364
33865901
34333441
34645963
34649194
34650330
34689348
34695064
34995963
8361994
9133356
9224908
9884913
9901669
10262615
10268059
10580036
10858989
11075787
11760770
11821707
11830253
11862119
11936642
11982430
12074751
12097291
12120023
12120483
12120525
12121011
12251007
14303206
14317094
15248209
16089401
16090586
16101389
16107870
16147759
16150161
16151011
16187182
16189407
16255777
17160506
17248563
17912254
20126863
20383682
21112445
21419070
22207618
23459027
23749096
23755220
23764933
23838335
23962968
23967945
23975115
23992089
24008084
24030922
24249966
24250281
24274814
24288904
24739741
24823394
25795308
26065894
26092592
26092603
26092617
26229638
26688926
26812092
26876849
27879287
27883220
27892026
28702992
28773967
28791280
28835856
28838346
28881426
28881458
28888268
29010153
29062161
29064754
29074340
29093179
Chrom. 7
301604
320403
805868
814038
826098
855737
1327352
1810606
1917474
1946070
2048597
2242338
2249065
2509514
3339014
3433287
3449405
3746492
3748218
3805367
4050803
4149279
4789081
4840777
5004635
5007217
5045399
5077349
5084808
5089381
5252132
5275128
5363679
5449080
5500743
9014407
9085792
9591840
9650990
9665743
9692745
9694189
9695252
9695380
9697869
10325731
10382166
10452451
11036687
11358921
11578601
11615616
11661181
12757217
12866588
13023345
13025693
13210100
13273118
13344769
13385528
13387036
13399507
13412225
13439227
13445771
13456350
13487751
13870607
13935768
13942865
13996490
14024835
14033564
14117072
14134663
14146913
14173371
14201994
14219853
14234292
14240796
14247995
14271330
14321603
14323858
14330634
14330767
14330805
14337208
14337819
14539304
14719071
14832870
14834287
14845995
14848549
14951905
14954513
14972459
15089740
15095800
15108657
15130615
15135188
15137472
15143187
15143894
15160572
15166127
15166295
15167737
15169012
15174864
15178156
15231331
15284520
15290117
15291269
15292396
15296100
15297427
15297686
15409629
15436364
15450801
15454819
15564620
15610517
15636705
15642588
15642903
15651826
15702282
16099788
16501998
16503815
16810810
16924331
16959947
16960117
16960276
16960401
16963960
17119746
17204584
17277388
17418071
17430463
17433914
17567314
17879716
17900863
17966477
18526987
20724566
21629066
21649639
22062321
22576585
25583207
25975729
26039205
26249224
26422355
26629955
26703705
26850748
26852789
26878501
26878892
26898808
26978699
26992355
26992412
26994142
27002696
27014135
27020531
27021504
27033840
27034127
27034234
27039439
27075704
27090685
27139249
27163747
27163759
27164035
27166549
27178113
27186177
27221765
27231920
27232125
27236531
27236655
27255358
27297867
27299956
27304944
27307241
27310870
27343972
27364470
27365842
27365964
27370083
27375270
27375530
27375560
27412304
27476404
27489012
27502405
27528418
27568155
27574240
27575855
27689645
27694641
27739292
27746745
27753430
27759841
27764841
27765340
27768432
27835693
27849579
27852031
27905229
27985925
27986934
28020458
28030569
28137794
28144761
28434849
28631917
28633531
28648562
28655230
28680207
28721912
28830284
28838337
28849611
28873399
28991186
29035494
29042565
29053147
29057299
29060096
29084206
29089416
29100516
29136860
29140920
29155892
29169638
29181761
29230537
29231018
29275722
29293526
29319719
29336514
29373429
29391586
29586631
29589488
29603732
29683974
29705972
29716030
29725178
29733163
29766798
29767776
29768163
29788127
29793765
29819960
29821716
29823771
29834797
29835194
29837347
29837909
29840523
29903316
29951331
30020450
30035913
30174433
30600816
30750076
31240794
31755760
31760878
31804014
31899399
31978333
32011670
32111494
32180983
32202940
33719215
33720269
33768667
33772893
33779125
33779145
33780159
33786357
33789038
33791167
33799415
33806822
33818932
33819177
33827615
33830177
33830879
33830893
33845848
34013561
34369414
34628190
34668103
34763890
34768013
34768328
34777346
34777527
34789894
34804546
34878772
35083027
35117028
35138979
35174823
35187791
35189129
35191878
35192053
35192244
35192646
35193429
35239555
35268907
35588617
35671777
35678918
35690497
35741628
35746287
35782964
35881172
35919518
35929780
Chrom. 8
1315450
1316987
1328771
1333065
1340577
1435941
1444029
2142734
2145885
2180880
2184342
2190909
2194059
2195528
2220029
2243267
2278279
2489494
2538223
3389346
3391519
3869323
3925538
4062838
4144247
4155720
4164789
4228455
4239665
4239919
4380845
4398015
4406052
4471233
4801008
4814319
5110731
5143652
5158428
5175323
5196330
5295668
5343692
5392051
5425106
5447981
5459887
5461620
5475342
5478810
5482853
5483511
5495371
5584834
5748196
5931455
6418464
6452425
6455275
6494189
6514722
6564935
6579803
6587439
6593317
6695179
6695743
6699698
6734037
7118046
7389775
7682227
7792972
7864606
7955742
8280426
8281327
8309231
8347824
8623057
8633970
8815878
8819436
8823728
8836505
8846676
8849095
8854448
8897308
8913345
8914897
8921949
8923085
8923493
8923505
8924452
8926268
8929363
8947803
8950850
8953466
8954865
8961610
8961700
8971068
8977561
9022379
9043225
9046732
9060315
9061786
9062163
9062962
9069728
9069870
9074589
9121687
9159524
9222702
9280159
9285950
9316310
9317334
9349202
9370444
9379097
9445719
9449022
9472748
9514012
9514518
9515768
9525496
9539376
11757553
11764690
11853505
12034744
12034887
12038696
12041295
12046363
12057567
12060573
12077571
12086219
12165189
12759165
12814625
12833774
12853568
12859772
12886487
12947280
13004113
13005356
13007264
13010623
13013410
13020243
13020300
13040043
13061222
13385617
13386443
13398828
13424383
13425577
13457848
13459374
13461869
13484138
13487126
13550031
13566761
13570318
13592246
13592389
13790741
13825307
13863769
13869761
14019516
14043362
14052736
14124519
14128407
14251037
14290666
14333053
14343568
14347928
14357273
14407854
14569753
14607129
14633046
14658309
14673156
14694593
14696791
14697333
14739332
15006690
15074407
15092546
15122845
15122872
15128963
15147660
15180898
15224275
15225572
15236047
15242711
15249727
15249921
15344380
15344852
15344866
15385963
15557503
15633092
15648285
15752324
15844315
15844879
16371706
16395854
16419162
16519466
16589375
16695574
16803434
16978869
17040597
17043881
17049562
17093324
17113293
17301564
17305138
17499125
17559959
17851871
17868537
18117693
18131477
19045996
19048518
19056865
19061528
19157894
19271385
19364744
19369077
19412196
19438572
19473613
19674489
19696513
19745549
19861059
19863283
19865558
19930686
20087896
20090937
20215311
20290592
20345348
20366169
20658566
21255415
21677713
21801515
22078898
22686555
23067462
24539406
25279378
25545503
25545900
25589279
25602384
25603723
25662527
25669389
26188686
26514724
26730999
26848534
26854707
26968050
27203252
27205849
27212112
27213527
27215141
27224983
27225392
27226092
27328888
27329483
28019431
28476279
28508017
28518667
28541862
28701017
28870217
28940378
29038803
29059957
29093169
29099235
29172343
29256419
29272607
Chrom. 9
1131852
1140397
1236198
1362899
1444994
1457947
1471518
1544205
1611822
1627546
1628368
1631244
1631468
1632895
1647627
1653688
1658589
1665457
1665964
1682294
1729930
1730631
1747927
1757168
1767075
1767132
1767189
1774923
1790547
1790770
1792585
1792696
1796973
1816611
1817024
1818202
1818766
1819391
1819457
1830782
1854190
1857209
1857522
1858092
1890619
1939255
1941976
1959702
1960710
1968608
1978438
1994293
1995543
2001243
2005597
2006607
2012109
2623770
2712837
2717556
2719421
2721055
2721836
2722595
2722856
2723778
2764984
2789030
2794079
2828159
2909018
2912494
2949226
2949450
3370301
3418015
3541738
3607916
3685573
3763705
4253872
4253878
4254162
4408727
4438907
4439225
4561179
4574782
4576406
4621502
4622747
4629131
4680132
5595471
5644055
5646321
5685010
5960545
5963044
5969327
6027797
6165184
6172452
6196065
6591135
6608302
6611099
6908032
7126161
7471942
7570379
7600464
7662255
7907466
7974284
7996541
7998780
7999415
8008492
8010476
8154590
8154634
8156598
8230151
8281432
8281443
8283134
8352750
8357487
8358754
8373632
8374536
8375090
8379835
8403633
8419586
8427362
8434608
8439274
8440453
8445813
8466575
8479123
8479246
8483060
8491710
8494363
8495971
8496986
8615412
9280192
9295553
9460081
9494132
9498792
9511309
9515502
9591055
9593650
9599799
9644753
9653489
9864856
9880379
9881305
9905138
10475669
10625990
10645249
10672930
10672938
10754378
10807294
10823684
10861281
10868266
11987274
12058144
12901654
13002733
13163498
13173429
13347989
13425070
13427496
13608199
13757110
14156750
14185203
14194773
14289974
14463910
14628095
15242003
15250109
15287949
15293390
15341820
15446995
15557669
15582428
15660852
15774230
16700369
16710836
16731182
16811311
16819626
17361368
17659538
17847921
17851246
17851648
18179118
18179225
19237779
19735122
19745125
20011248
21055992
21421796
21494626
21774684
22009352
22010245
22050224
22056779
22074536
22387551
22555515
22685443
22693262
22698457
22703007
22703066
22896679
22962150
23298083
Chrom. 10
1495914
801341
866499
968395
1350919
1462850
1609501
1683660
1719622
1914635
2090260
2119292
2186376
2240597
2277641
2277755
2302370
2333605
2333609
2604032
2772386
2802715
2803847
2823201
2824311
2844753
2848203
2849767
2851840
2857010
2864180
2877295
2917390
2917746
2991274
3221516
3329587
3655013
3817963
4401887
4527548
5240401
6254070
6285147
6296424
6358155
6373115
6405418
6412805
6424580
6611317
6682737
6713636
6723870
6791880
6808688
6823704
6846208
6853382
6918143
6925317
6932411
6937763
6955809
6957849
6962213
7421331
7421385
7508499
7605752
7669584
7680492
7684775
7691610
7705076
7708600
7767102
7945551
7958502
7972061
8501730
8712363
8743690
8744138
8744316
8770467
8898004
8956013
9079134
9088412
9100952
9106742
9106763
9130046
9130175
9135222
9175618
9199783
9285137
9701297
9723630
9728149
9967973
9984099
10038430
10040283
10161735
10315219
10678185
10763460
11145709
11180297
11183679
11233813
11362740
11391812
11425184
11506271
11818149
12185748
12185793
12189467
12214463
12232419
12321116
12409569
12536882
12549842
12595775
12598176
12598853
12601339
12684821
12810954
13213072
13220134
13282947
13319446
13354511
13387814
13447134
13472869
13761009
14006299
14208307
14219109
14266594
14297099
14523767
14570377
14570429
14667689
14740902
14741617
14745710
14746384
14754379
14758510
14759237
14763048
14786627
14788655
14792354
14811735
14812739
14818072
14821963
14838025
14891750
14892276
14896510
14932274
14972614
15023789
15141800
15144334
15220584
15392718
15394359
15527611
15541212
16609829
16616818
16842942
16856719
16937814
16942287
17904460
18492514
18674710
18678799
19061304
19454535
19457157
19457917
19458128
19999395
Chrom. 11
11351199
891300
1581770
1600067
1683918
1686571
1728743
1863208
1906700
1911888
2056187
2063197
2081284
2138828
2155195
2224652
2225923
2228119
3969220
4291412
4463364
4494342
4622583
4655945
4715967
4833405
4837700
5151149
5940706
7647894
7761730
7801704
7896980
7908053
8115329
8115768
8116236
8409843
8538463
8962219
9139740
9384878
9397891
9652177
10092795
10320291
10361018
10381934
10402877
10487159
10960086
11218443
11240157
11280838
11283151
11283389
11293534
11297518
11299037
11318104
11322256
11352184
11367752
11582590
11619742
11621886
11622879
11628550
11629227
11641156
11644450
11647778
11650702
11650769
11653804
11663170
11689525
11693345
11694209
11702974
11707148
11716714
11968452
12106514
14618722
14704540
14832419
14973901
15067082
15275895
15828549
15829930
15956238
16114058
16205941
16591272
16643137
16825614
16919794
16972201
18141309
18141324
18143571
18175001
18189043
18459577
18503235
18767161
18768975
18768986
18772517
18795180
18795189
18864943
19178977
Chrom. 12
51732
176426
276209
397384
398610
450553
521880
542510
542557
563307
661095
661440
661517
661665
662661
690015
695529
697750
698223
708358
720138
733457
736291
737246
740337
742588
748792
752453
754563
763364
769021
769965
799588
1027253
1035358
1042407
1042944
1165509
1174170
1175485
1212203
1272418
1329079
1329520
1360707
1371634
1374116
1377862
1432310
1490269
1509999
1543062
1621397
1636027
1656909
1665995
1687066
1687135
1691864
1699838
1700768
1703169
1706337
1736433
1788357
2244294
2446121
2457549
2458438
2594356
2607630
2687627
2747914
2762515
3335463
3377283
3382627
3479415
3575255
3656884
3689679
3700933
3711136
3712025
3719080
3726040
3726438
3727589
3822633
3928204
4010287
4036336
4116420
4247390
4260509
4460565
4552300
4562948
4685899
4686340
4686578
4824581
4868813
4880409
4889641
4957095
4976981
4977763
4979539
4979706
5302382
5399238
5449655
5472277
5497341
5529097
5619289
5625183
5636033
5795446
5826085
5873446
5873556
5898603
5903821
6210848
6212759
6279058
6304121
6305062
6306552
6334170
6337183
6342556
6352811
6367810
6369556
6370024
6379514
6385026
6387502
6393292
6520842
6521009
6943359
6944328
6969420
7012484
7025099
7086951
7722653
7824724
8482351
8757625
9217309
9277691
9399852
9428945
9436091
9437805
9567478
9697070
10122859
10394296
10423289
10512222
10609404
10858472
10880311
11730907
11783849
11787324
11824369
11856004
11905675
11924333
11971411
12011879
12011888
12021455
12021659
12024511
12027892
12477335
12485199
12491940
12511016
12521397
12545271
12546969
13485946
13509694
13538909
14177283
14225420
14427210
14554492
14789914
15260838
15388234
15542923
16026339
16132562
16419180
16425162
16425264
16730119
16787346
17292280
17293207
17335011
17347985
17363790
17685277
17685380
17784817
17917172
18165884
18555650
18565669
18568400
18568854
18596275
18596396
18691310
18694275
18709215
18709519
19186431
Chrom. 13
754274
893530
1896710
1981964
2443603
2521595
2832242
2870925
3285202
3745707
3745722
4611051
4655648
5408399
5439104
5444321
5445388
5493049
5695438
5700866
5965190
6050483
6137657
6137673
6199135
6265908
6271195
6290088
6295751
6306927
6347591
6347626
6486823
6565794
6706060
6727085
6729058
6738588
6888646
7170932
7331658
7827138
8401337
8567731
8938041
9012796
9023548
9544248
9888714
10526785
10660063
11026377
11028713
11075965
11705116
11709529
11792287
11822391
11934618
11990165
12021075
12045999
12057725
12103441
12103706
12104389
12111103
12116512
12138572
12155761
12161905
12226537
12238754
12240815
12257109
12275710
12298652
12759552
13374481
13475246
13575463
14066325
14510508
14510801
14890912
14989832
15861035
16008059
16065472
16251932
16399761
16627002
17092078
17133072
17183261
17330579
17373832
17426611
17428374
17434757
17445579
17453099
17454605
17482809
17494234
17495930
17515969
17872572
17873691
17925090
18052596
Chrom. 14
1138641
1184277
1477415
1540679
1543547
2081096
2106988
2265777
2279076
2304780
2324654
2330596
2331106
2331114
2334011
2436344
2445621
2504379
2534559
2863000
2877893
2888580
2900733
2919741
2926803
2931291
2932764
2954320
2958241
2958371
3026314
3092581
3097743
3108633
3192556
3385119
3404797
3428785
3442312
3451277
3455949
3615271
3625853
3639827
3665270
3666147
3720087
3722871
3726177
3727676
3789566
3790879
3806797
3942165
3966352
3974602
4592247
4592785
4610605
4630776
4633141
4648452
4662019
4741756
4751778
4769384
4807786
4913952
4914002
4950032
4959128
4961156
5000387
5001220
5011687
5019119
5056515
5234564
5283059
5331820
5361858
5367483
5369311
5413765
5479705
5482244
5508343
5511388
5518179
5547996
5555946
5841809
5983642
6370002
6595300
6596177
6600169
6740436
6753103
6785274
6947447
7208751
7209342
7210856
7276970
7310590
7336448
7344432
7398658
7493466
7524051
7880754
7893701
8059214
8059226
8062902
8071662
8071665
8099475
8115429
8126869
8127796
8128165
8133352
8142040
8142769
8148118
8162012
8210184
8217810
8940474
9595312
9595609
10025397
10106277
10335114
10399461
10413814
10476927
10508240
10517012
10552049
10653992
10668907
10992692
11960776
12117345
12253079
12413874
12433516
12721749
12852505
12853507
12888750
12925013
12940572
12944800
12944881
12948156
13062440
13531394
13556120
13574609
13980135
Chrom. 15
2194376
2232471
2666055
3313219
4173586
4627060
4633267
4710758
4850805
4978411
4978416
5072570
5729117
5856302
5952754
5986957
5992373
6825004
6840484
6890928
6997784
7005312
7006565
7008208
7021225
7023259
7023729
7237941
7401141
7655098
7707832
7732764
7736267
7748539
7748588
7913333
7926598
8001138
8003013
8003087
8391874
8883186
9149389
9249964
9335909
9388136
9397019
9502584
9506460
9512881
9524442
9525027
9600201
9643276
9648925
9662551
9707692
9742644
9742743
9749487
9750184
9760273
9760932
9841882
9855081
9941258
9842218
9953346
9997920
10181612
10245041
10305483
10307245
10310536
10347798
10395261
10412623
10472809
10730490
11329951
11396849
11396869
11399138
11400087
11470350
11470490
11470659
11764691
11795592
11916147
12051325
12073158
12073681
12074970
Chrom. 17
863806
906851
1098618
1439118
1442589
1451834
1569753
1592010
1661517
1724848
1725150
1747282
1759899
1781309
1804137
2391148
2395880
2474904
2581730
2649003
2739230
2780424
2782074
2788073
2788693
2803990
3120122
3143043
3168127
3176559
3185391
3263636
3730560
3730704
3810149
3819704
3824304
3825479
4075070
4144297
4200040
4218760
4343840
4853626
4881769
4913138
4918927
5194029
5653398
5770999
5952417
6340367
6559597
6573224
6588610
6588877
6611979
6700041
6722116
6774639
6775595
6798651
6817223
6831105
7100372
7106078
7181421
7181450
7209913
7265946
7291454
7291475
7404183
7463775
7504942
7540746
7555401
7617800
7638790
7644886
7780001
7864358
7901422
7982966
8000081
8054792
8218316
8305821
8321755
8343020
8344420
8347773
8386505
8410360
8427145
8576407
8592228
8607586
8627534
8632093
8638248
8654757
8655022
8655179
8658931
8660545
8690000
8694078
8717538
8722184
8787695
8992268
9105384
9106322
9123913
9141589
9146850
9149226
9149263
9396157
9397450
10025334
10190550
10228462
10265575
10320830
10903863
Chrom. 18
902645
977710
1093065
1124752
1165817
1208234
1209138
1231936
1256978
1522336
1529107
1543545
1550632
1590860
1600690
1639026
1706460
1798888
1859042
2179391
2553528
2870516
2885180
2895564
2914143
2950413
2951088
3182918
3274870
3285062
3286431
3289038
3306804
3307774
3406039
3422606
3550356
3575439
3796117
4057707
4089443
4104363
4133004
4133737
4134351
4134920
4135566
4138386
4273700
4296051
4583312
4749214
4808924
4841700
4903508
4913686
5039644
5517339
5522749
5602842
5602848
6141471
6189234
6252403
6633350
6636041
7358034
7527461
7540992
7547309
7574920
8520358
8892020
9491434
9521363
9521864
9984590
9987506
10010477
10267891
Chrom. 19
6735711
6760809
6818457
6992754
171630
271482
580436
747638
838192
876247
1003675
1408165
1502032
1505279
1539817
1547313
1569669
1580462
1751180
1796257
1796923
1809408
1876738
1985088
2259820
2356804
2434748
2619904
2641336
2845568
3550591
3585622
3651040
3651331
3652052
3678657
3678661
3687105
3759136
3759162
3766184
3779485
3779599
3782009
3814901
3939895
3962214
4000300
4063257
4372316
4638239
4724668
5451419
5465889
5500154
5726543
5747298
5747488
5830696
5833728
5833753
5841962
5891453
5894315
5965904
5989183
5989235
6010174
6020007
6022617
6025436
6027194
6047730
6054505
6095664
6095884
6096481
6100849
6101015
6109977
6118616
6161955
6371540
6527535
6531519
6549159
6597719
6602651
6673957
6702522
7189659
7194757
7194939
7195075
7200145
7203329
7224289
7417096
7430290
7435989
7532028
7544763
7583548
7604030
7633191
7669414
7677916
7757436
8014377
8071886
8259175
8300311
8416214
8434392
9303967
Chrom. 20
625393
813936
837059
911318
927790
971372
974467
974560
978996
989001
993560
1000718
1004332
1073683
1395259
1404631
1412280
1419154
1460398
1530162
1532476
1538650
1561203
1626986
1758206
1759384
1801935
2305416
2377369
2426602
2477792
2568813
2569043
2668097
2677844
2836739
2900096
2936787
2982291
2982302
2995437
3130379
3181759
3199954
3201300
3787277
4507226
4922176
5358625
6017404
6146979
6460722
6484087
7157261
7305471
8111695
8191456
8408068
8412195
8443839
8489417
8582641
8789679
8931031
8938913
8944640
9254545
9271756
9286346
9294047
9325396
9805264
9849284
10114240
10285739
10508386
10763997
10764208
10930631
10959899
10991662
11075418
11505812
11676390
11814885
11824650
11830800
12066976
12075797
12186799
12268210
12670983
12671317
12687999
12716334
12761427
12837139
12861404
12892794
12905265
12923319
12946708
13087908
13207717
13214456
13215531
13216111
13334571
13344724
13364176
13483320
1393885
Chrom. 21
433173
912762
1029141
1390021
1554352
1641909
1653319
1653357
1831102
2333072
2469788
2559790
2610337
2618503
2624195
2688709
2832321
2974147
2994600
3001665
3005099
3024714
3516402
3528476
3536570
3558629
3684754
3686478
3723987
4054918
4836900
4939003
4948026
4976109
4997453
5022715
5126628
5291715
5314232
5323538
5678012
5759162
5762368
5762381
5764164
6571837
6571938
6576343
6580599
Chrom. 22
1663230
2100481
2547055
2552848
2729474
2798914
3700626
3703666
3704248
3716675
Chrom. 23
303275
541486
573262
573539
584175
609173
616284
648063
660965
663857
664421
738474
772230
775923
792876
804158
808523
829432
853467
873415
874662
882886
1002478
1045090
1156676
1531846
1577757
1614192
1615332
1637697
1644570
1657014
1659546
1929541
1948145
2008332
2410540
2686617
2704778
2769886
2950084
3111000
3272938
3368358
3772854
4450830
4450876
4470184
4567627
4884561
4916097
Chrom. 24
126822
149303
200583
284769
1405250
1421182
1912123
1986908
1988653
2059417
2059419
2097033
2123113
2145047
2330171
2330498
2652186
2659757
2688398
2872478
2873532
2875411
2922961
2924593
2992122
3211402
3211739
3217940
3225884
3231008
3494353
3496578
3572763
3636773
3666284
3758125
3863025
4332717
4459949
4769482
5102087
5890836
6037678
Chrom. 25
978009
1102452
1103192
1109695
1150999
1320004
1328120
1528888
1669503
1704211
1704246
1778485
1856815
2053338
2379274
2492072
2534366
Chrom. 26
382874
504218
506657
509283
528461
543884
550031
569259
588357
602694
699097
713348
715959
775775
808129
808162
828736
1121275
1941887
2225324
2282561
2591052
2621536
2773637
2820275
2874261
2876457
2876551
2880150
2888638
2890690
2999174
3010775
3013540
3045860
3073425
3103563
3115215
3115232
3129055
4772959
4976868
Chrom. 27
1432737
1435380
1436167
1445658
1454549
1457215
1490347
1490619
1564845
1565230
1570143
1571190
1582033
1591385
1594214
1596504
1596914
1601776
1603418
1609162
1613400
1619962
1619971
1619989
1620516
1625362
1626371
1639263
1909749
1949283
2053097
2053967
2156431
2752990
2927799
2966444
3052200
3195680
3458427
3458886
3475696
3551689
3632525
3637822
3875240
3988099
4038299
4139773
Chrom. 28
151802
151819
1062404
1957082
2443298
2641875
2675966
2791367
2835337
2912314
2935182
3218402
3270473
3491291
3501746
3548523
3593309
3623347
3635463
3878808
3907769
3985591
3985871
Chrom. 33
414716
599116
1461466
1485829

Unlocalised Scaffolds
chr1_NT_456097v1_random: 43874
chr1_NT_456113v1_random: 3429
chr1_NT_456143v1_random: 7614
chr1_NT_456856v1_random: 5170
chr1_NT_457819v1_random: 308
chr13_NT_462202v1_random: 5933
chr14_NT_462351v1_random: 19241
chr14_NT_462351v1_random: 19095
chr3_NT_459020v1_random: 12054
chr3_NT_459020v1_random: 118744
chr3_NT_459020v1_random: 64013
chr3_NT_459020v1_random: 106471
chr3_NT_459020v1_random: 64289
chr3_NT_459020v1_random: 13514
chr4_NT_459801v1_random: 31636
chr4_NT_459801v1_random: 166828
chr5_NT_460430v1_random: 18341
chr5_NT_460548v1_random: 7262
chr6_NT_460840v1_random: 23366
chr22_NT_463002v1_random: 9929
chr33_NT_463452v1_random: 15025
chr33_NT_463454v1_random: 57531
chr33_NT_463456v1_random: 36158
chrLGE64: 868044
chrUn_NT_464128v1: 67946
chrUn_NT_464135v1: 21542
chrUn_NT_464148v1: 5652
chrUn_NT_464215v1: 7282
chrUn_NT_464327v1: 17129
chrUn_NT_464492v1: 18892
chrUn_NT_465142v1: 16062
chrUn_NT_465200v1: 5520
chrUn_NT_466029v1: 6006
chrUn_NT_466029v1: 3712
chrUn_NT_466086v1: 822
chrUn_NT_466086v1: 8623
chrUn_NT_467376v1: 1658
chrUn_NT_467869v1: 10766
chrUn_NT_469469v1: 2442
chrUn_NT_469469v1: 2838
chrUn_NT_471611v1: 6225
chrUn_NT_473432v1: 5412
chrUn_NT_473478v1: 911
chrUn_NT_473624v1: 3973
chrUn_NT_473988v1: 822
chrUn_NT_479485v1: 871
chrUn_NT_479485v1: 432

1. A method of certifying a test animal-derived product sample, the method comprising the steps of:
(a) determining a test methylation profile of one or more pre-selected methylation sites within the genomic material obtained from the test animal derived product sample; and
(b) comparing the test methylation profile obtained from (a) with a reference methylation profile obtained from a control animal of the same biological taxon of the test animal from which the product sample is derived from, where the control animal is not slaughtered by a single cut across the throat severing both carotid arteries, both jugular veins, both vagus nerves, trachea and/or oesophagus and/or the control animal is not bled to death
wherein a difference in the test methylation profile of (a) compared to the reference methylation profile from the control animal, is indicative of the test animal having been slaughtered by a single cut across the throat severing both carotid arteries, both jugular veins, both vagus nerves, trachea and/or oesophagus and/or of the test animal having been bled to death and the test animal-derived product is certified so; and
wherein the pre-selected methylation sites are CpG sites selected from genes or regions of genomic DNA from the control and test animal that shows the highest degree of methylation variation during the training of the method.

2. A method of certifying a test animal-derived product sample, the method comprising the steps of:
(a) determining the test methylation profile of one or more pre-selected methylation sites within the genomic material obtained from the test animal derived product sample; and
(b) comparing the test methylation profile determined in (a) with a panel of predetermined reference methylation profiles of the same biological taxon of the test animal from which the product sample derives, wherein each of the predetermined reference methylation profiles is specific for a distinctly certified reference animal that
has been slaughtered by a single cut across the throat severing both carotid arteries, both jugular veins, both vagus nerves, trachea and/or oesophagus; and/or
bled to death;
wherein if the test methylation profile is significantly similar to one of the predetermined reference methylation profiles, the test animal derived product sample has the same distinct certification as that of the predetermined reference methylation profile and is derived from a test animal that has been slaughtered by a single cut across the throat severing both carotid arteries, both jugular veins, both vagus nerves, trachea and/or oesophagus and/or bled to death and the test animal-derived product is certified so; and
wherein the pre-selected methylation sites are CpG sites selected from genes or regions of genomic DNA from the control and test animal that shows the highest degree of methylation variation during the training of the method.

3. A method of certifying a test animal-derived product sample, the method comprising the steps of:
(a) determining the methylation status of at least one CpG site within the genomic material obtained from the animal-derived product sample,
(b) comparing the methylation status of the CpG site from (a) with that of a control animal of the same biological taxon of the test animal from which the product sample is derived from, where the control animal is not slaughtered by a single cut across the throat severing both carotid arteries, both jugular veins, both vagus nerves, trachea and/or oesophagus and/or the control animal is not bled to death,
wherein a difference in the test methylation status of (a) in the test animal compared to the CpG site in the control animal is indicative of the test animal having been slaughtered by a single cut across the throat severing both carotid arteries, both jugular veins, both vagus nerves, trachea and/or oesophagus and/or of the test animal having been bled to death and the test animal-derived product is certified so.

4. The method according to claim 1, wherein test animal derived product sample is distinctly certified kosher or halal where the test animal is slaughtered by a single cut across the throat severing both carotid arteries, both jugular veins, both vagus nerves, trachea and/or oesophagus and/or the test animal is bled to death.

5. A method of verifying certification of a test animal-derived product sample that has been certified to derive from a test animal that was slaughtered by a single cut across the throat severing both carotid arteries, both jugular veins, both vagus nerves, trachea and/or oesophagus and/or that was bled to death, the method comprising the steps of:
(a) determining a test methylation profile of one or more pre-selected methylation sites within the genomic material obtained from the test animal derived product sample; and
(b) comparing the test methylation profile obtained from (a) with a reference methylation profile obtained from a control animal of the same biological taxon of the test animal from which the product sample is derived from, where the control animal is not slaughtered by a single cut across the throat severing both carotid arteries, both jugular veins, both vagus nerves, trachea and/or oesophagus and/or the control animal is not bled to death
wherein a difference in the test methylation profile of (a) compared to the reference methylation profile from the control animal, is indicative of the test animal having been slaughtered by a single cut across the throat severing both carotid arteries, both jugular veins, both vagus nerves, trachea and/or oesophagus and/or of the test animal having been bled to death and the test animal-derived product being correctly certified, and wherein a similarity in the test methylation profile of (a) compared to the reference methylation profile of the control animal, is indicative of the test animal not having been slaughtered by a single cut across the throat severing both carotid arteries, both jugular veins, both vagus nerves, trachea and/or oesophagus and/or of the test animal having been bled to death and the test animal-derived product being wrongly certified; and wherein the pre-selected methylation sites are CpG sites selected from genes or regions of genomic DNA from the control and test animal that shows the highest degree of methylation variation during the training of the method.

6. A method according to claim 1, wherein the CpG site is selected from list provided in Table A.

7. A method for confirming or rejecting an assumed certification of an animal derived product sample, the method comprising
comparison of a test methylation profile obtained from genomic material of the animal derived product sample with a reference methylation profile obtained from a control animal of the same biological taxon of the test animal from which the product sample is derived from, where the control animal is not slaughtered by a single cut across the throat severing both carotid arteries, both jugular veins, both vagus nerves, trachea and/or oesophagus and/or the control animal is not bled to death,
wherein a difference in the test methylation profile compared to the reference methylation profile from the control animal, is indicative of the test animal having been slaughtered by a single cut across the throat severing both carotid arteries, both jugular veins, both vagus nerves, trachea and/or oesophagus and/or of the test animal having been bled to death
wherein the assumed certification is that the test animal derived product sample is kosher or halal where the test animal is slaughtered by a single cut across the throat severing both carotid arteries, both jugular veins, both vagus nerves, trachea and/or oesophagus and/or the test animal is bled to death.

8. A method of determining if a test animal-derived product sample, is obtained from a test animal that has been bled to death or slaughtered by a single cut across the throat severing both carotid arteries, both jugular veins, both vagus nerves, trachea and/or oesophagus the method comprising:
comparison of a test methylation profile obtained from genomic material of the test animal derived product sample with a reference methylation profile obtained from a control animal of the same biological taxon of the test animal from which the product sample is derived from, where the control animal is not slaughtered by a single cut across the throat severing both carotid arteries, both jugular veins, both vagus nerves, trachea and/or oesophagus and/or the control animal is not bled to death.

9. The method according to claim 8, wherein the method comprises the steps of:
a) determining the test methylation profile of one or more pre-selected methylation sites within the genomic material obtained from the test animal derived product sample; and
b) comparing the test methylation profile determined in (a) with a panel of predetermined reference methylation profiles of the same biological taxon of the test animal from which the product sample derives, wherein with a reference methylation profile obtained from a control animal of the same biological taxon of the test animal from which the product sample is derived from, where the control animal is not slaughtered by a single cut across the throat severing both carotid arteries, both jugular veins, both vagus nerves, trachea and/or oesophagus and/or the control animal is not bled to death,
wherein a difference in the test methylation profile of (a) compared to the reference methylation profile from the control animal, is indicative of the test animal having been slaughtered by a single cut across the throat severing both carotid arteries, both jugular veins, both vagus nerves, trachea and/or oesophagus and/or of the test animal having been bled to death.

10. The method according to claim 1, wherein the animal is selected from the group consisting of cow, sheep, goat, camel, chicken, goose, duck and turkey.

11. The method according to claim 1, wherein the test animal-derived product sample is meat, eggs, milk and any other tissue or sample that provides genomic DNA.

12. The method according to claim 1, wherein the test animal-derived product sample is meat.

13. The method according to claim 1, wherein the difference is hypomethylation or hypermethylation.

14. Use of DNA methylation profiling for certifying of an animal-derived product sample,
wherein the certification of the animal-derived product sample is kosher or halal where the test animal is slaughtered by a single cut across the throat severing both carotid arteries, both jugular veins, both vagus nerves, trachea and/or oesophagus and/or the test animal is bled to death.
